(19) United States
(12) Patent Application Publication
(10) Pub. No.: US 2024/0010604 A1
(43) Pub. Date: Jan. 11, 2024

(54) OPTIMIZED PROCESS FOR SYNTHESIZING METHACRYLIC ACID (MAA) AND/OR ALKYL METHACRYLATE BY REDUCING UNWANTED BYPRODUCTS

(71) Applicant: Röhm GmbH, Darmstadt (DE)

(72) Inventors: Steffen Krill, Muehltal (DE); Florian Klasovsky, Haltern am See (DE); Daniel Helmut König, Stuttgart (DE); Patrick Wings, Koeln (DE)

(73) Assignee: Röhm GmbH, Darmstadt (DE)

(21) Appl. No.: 18/250,207

(22) PCT Filed: Oct. 19, 2021
(86) PCT No.: PCT/EP2021/078866

§ 371 Date: Apr. 22, 2023

(30) Foreign Application Priority Data
Oct. 23, 2020 (EP) 20203724.8

(51) Int. Cl. C07C 67/20; C07C 51/06; C07C 67/54; C07C 51/44

(52) U.S. Cl. CPC C07C 67/20; C07C 51/06; C07C 67/54; C07C 51/44

(57) ABSTRACT

An improved process for synthesizing methacrylic acid and/or alkyl methacrylates, in particular methyl methacrylate (MMA), involves reacting acetone and hydrogen cyanide in the presence of an alkaline catalyst in a first reaction stage such that a first reaction mixture containing acetone cyanohydrin (ACH) is obtained. The process then involves working up the first reaction mixture containing acetone cyanohydrin (ACH), reacting acetone cyanohydrin (ACH) and sulfuric acid in a second reaction stage (amidation), and heating the second reaction mixture in a third reaction stage (conversion), such that methacrylamide (MAA) is obtained. The process further involves hydrolyzing or esterifying methacrylamide (MAA) with water and, optionally, alcohol, preferably water and optionally methanol, in a fourth reaction stage such that methacrylic acid or alkyl methacrylate is formed. The sulfuric acid used has a concentration of 98.0 wt % to 100.0 wt %.

The present invention relates to an improved process for preparing methacrylic acid (MA) and/or alkyl methacrylates, especially methyl methacrylate (MMA), comprising the reaction of acetone and hydrogen cyanide in the presence of a basic catalyst in a first reaction stage to obtain a first reaction mixture comprising acetone cyanohydrin (ACH), the workup of the first reaction mixture comprising acetone cyanohydrin (ACH), the reaction of acetone cyanohydrin (ACH) and sulfuric acid in a second reaction stage (amidation), and the heating of the second reaction mixture in a third reaction stage (conversion) to obtain methacrylamide (MAA), and the subsequent hydrolysis or esterification of methacrylamide (MAA) with water or with alcohol and water, preferably methanol and water, in a fourth reaction stage to form methacrylic acid or alkyl methacrylate, wherein the sulfuric acid used has a concentration in the range from 98.0% by weight to 100.0% by weight.

More particularly, the present invention relates to an optimized process for preparing methacrylic acid and/or alkyl methacrylate, comprising the specific adjustment and monitoring of the quality of the intermediates and products, especially MAA and MMA, wherein the formation of troublesome by-products, especially methacrylonitrile (MAN), acetone, methyl isobutyrate (MIB) and methyl propionate (MP), and also diacetyl (di-AC, butane-2,3-dione), in the precursors and intermediates is reduced, and the yield of intermediates and products is improved.

### PRIOR ART

Methyl methacrylate (MMA) is used in large amounts for preparing polymers and copolymers with other polymerizable compounds. Furthermore, methyl methacrylate is an important monomer for various specialty esters based on methacrylic acid (MA), which can be prepared by transesterification of MMA with the appropriate alcohol or are obtainable by condensation of methacrylic acid and an alcohol or amino alcohol. There is consequently a great interest in very simple, economic and environmentally friendly processes for preparing this starting material.

The preparation of methacrylamide by the amidation of acetone cyanohydrin (ACH) is a widely employed process. For example, such an amidation, which is also referred to as ACH hydrolysis in the prior art, is an important intermediate step in the preparation of methyl methacrylate by what is called the ACH-sulfo process, wherein large amounts of sulfuric acid are used in the process. The preparation of methacrylic acid (MA) and methyl methacrylate (MMA) by the ACH-sulfo process is common knowledge and is described in the prior art, for example WO 2008/068064, WO 2013/143812, EP 0 226 724. In this process, in a first step, acetone cyanohydrin (ACH) is first prepared by reaction of hydrogen cyanide and acetone, which is then converted to methacrylamide (MAA). These steps are described in U.S. Pat. No. 7,253,307, EP 1 666 451 or EP 2007 059092 inter alia. The conversion of ACH to MAA (amidation) is typically brought about by a reaction between ultra-highly concentrated sulfuric acid, especially oleum, and ACH. The reaction is exothermic, and so the heat of reaction is preferably removed rapidly from the system.

The conversion to MAA typically proceeds in two process steps. First of all, in the amidation step, an essentially anhydrous sulfuric acid solution comprising mainly alpha-hydroxyisobutyramide (HIBAm), the sulfate ester thereof alpha-sulfoxyisobutyramide (SIBA), and methacrylamide (MAA) (or protonated in salt form, in the form of the respective hydrogensulfates). In the subsequent step, called the conversion, this solution is typically converted to methacrylamide (MAA) with β-elimination of water or sulfuric acid at high temperatures between 130° C. and 200° C. with usually short delay times, for example about 20 min or less. Typically, after the conversion, the main MAA product is present with a concentration in the solution of about 30% by weight to 40% by weight (according to the sulfuric acid excess used).

The steps of amidation and of conversion, in terms of process technology, generally differ significantly in delay time and also in the temperature level used. With regard to the chemical reaction, the amidation is typically conducted for a shorter period than the conversion and typically at lower temperatures than the conversion.

Document U.S. Pat. No. 4,529,816 describes a process for preparing alkyl methacrylates by the ACH-sulfo process, wherein the amidation is performed at temperatures around 100° C. with substantially superstoichiometric amounts of sulfuric acid (molar ratio of ACH:H₂SO₄ of about 1:1.3 to 1:1.8). It is pointed out that the sulfuric acid used should contain sufficient SO₃ to assure at least an acid strength of 98%, preferably at least 99.3% More particularly, fuming sulfuric acid (oleum) should be used, which preferably has 101% sulfuric acid and hence free SO₃. In the subsequent step of the esterification, the reaction mixture is treated with an excess of water and alcohol at 100 to 150° C.. U.S. Pat. No. 4,529,816 states that the methyl alpha-hydroxyisobutyrate (MHIB) by-product is separated from the methyl methacrylate product after the esterification and recycled into the process. In a similar manner, in the process according to U.S. Pat. No. 5,393,918, by-products including methyl alpha-hydroxyisobutyrate (MHIB) are isolated, dehydrated and recycled into the reaction. It is stated that the concentration of sulfuric acid in the reaction of ACH is not crucial and is within the range from 95% to 100%.

Document DE 38 28 253 A1 describes a process for recycling spent sulfuric acid in the preparation of methacrylic esters by the ACH-sulfo process, wherein the spent acid, after the esterification, is concentrated, mixed with fresh acid and recycled. DE 38 28 253 A1 generally describes an acid strength of 96% to 101% in the reaction of acetone cyanohydrin with sulfuric acid.

Document DE 1 618 721 describes the reaction of acetone cyanohydrin (ACH) with sulfuric acid in two stages with a different ratio of sulfuric acid to ACH, by means of which the viscosity of the reaction mixture is to be controlled. In the process described in EP 0 226 724, the reaction is performed in the presence of an alkane solvent in order to control the viscosity of the reaction mixture and enthalpy of reaction.

Document CH 239749 describes a process for preparing methacrylamide by the action of sulfuric acid on acetone cyanohydrin at temperatures of 110-130° C. and 115-160° C., wherein 100% sulfuric acid, for example, is used.

As an alternative to the reaction of MAA solutions in sulfuric acid with alcohols and water to give alkyl methacrylates, the MAA solution in sulfuric acid obtained after the conversion can also be reacted with water to give methacrylic acid.

U.S. Pat. No. 4,748,268 describes a process for esterifying methacrylic acid with a C₁-C₄ alcohol in the presence of a high-boiling organic liquid in a plug-flow reactor, in which the reaction mixture is continuously fractionated, wherein the distillate stream has a relatively high proportion of methacrylic ester and the bottom stream is recycled predominantly into the plug-flow reactor.

By-products formed in the amidation and conversion include carbon monoxide, acetone, sulfonation products of acetone, and cyclocondensation products of acetone with various intermediates. These by-products mentioned can usually be separated relatively effectively from the alkyl methacrylate product. In addition, however, depending on the reaction conditions, other by-products are formed, the separation of which from the alkyl methacrylate, especially from the methyl methacrylate product, is difficult or associated with considerable separation complexity. For example, the separation is found to be difficult on account of the azeotrope boiling points and the boiling points of the specific compounds. These troublesome by-products are especially methacrylonitrile (MAN), acetone, methyl isobutyrate (MIB) and methyl propionate (MP), and also diacetyl (di-AC, butane-2,3-dione). Some of these troublesome by-products are responsible to a crucial degree for an elevated colour number in the alkyl methacrylate end product, especially MMA. Especially the removal of diacetyl has to be effective and preferably complete, firstly because not inconsiderable amounts are formed during the reactions, and secondly because the substance makes a crucial contribution to the colour number in the final MMA end product if it is detectable in ppm concentrations in the product.

These and other troublesome low molecular weight by-products may additionally cause problems in the course of further polymerization and processing of the polymers, for example as a result of outgassing during extrusion or in injection moulding. Troublesome by-products having a double bond are polymerized into the polymer product as well as the alkyl methacrylate and impair the properties of the polymers, for example transparency. In order to obtain on-spec alkyl methacrylate end product, especially MMA, the level of these by-products, such as MAN, MIB and/or MP, must be reduced in the reaction steps or they must be removed in the workup.

Methacrylonitrile (MAN) is typically formed as a by-product during the amidation reaction and in the SIBA elimination to give MAA in the conversion from acetone cyanohydrin (ACH) with elimination of water.

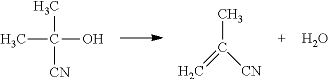

Methacrylonitrile (MAN) forms azeotropes both with methanol (MeOH) and with other substances present in the system, and can be separated from MMA azeotropes (for example the azeotropes with water and methanol) only with a not inconsiderable level of complexity on account of similar boiling points of the product. Furthermore, MAN, with regard to mixture properties and its polarity, behaves similarly to MMA and is therefore separable from MMA in extraction steps and phase separation apparatuses only with difficulty in these operations.

Complete removal of MAN with a reasonable level of cost and complexity is generally impossible and requires special measures.

What is especially desirable in this connection is to find means that also permit influencing of the formation, specifically a reduction in the formation, in the various reaction steps.

The amidation affords, as desired main products from the reaction, sulfoxyisobutyramide hydrogensulfate (SIBA*H₂SO₄) and methacrylamide hydrogensulfate (MAA*H₂SO₄) as a solution in excess sulfuric acid. Typically present additionally in the amidation solution is alpha-hydroxyisobutyramide hydrogensulfate (HIBAm·H₂SO₄), for example with a yield based on ACH of <5%. In the case of virtually full conversion of ACH, it is possible to obtain a yield of the above-described intermediates in the amidation step of typically about 94% to 96%. In this step, the above-described by-products are often formed in considerable amounts.

In addition, tar-like solid condensation products separate out of the ammonium hydrogensulfate- and sulfuric acid-containing process acid which is often regenerated in a sulfuric acid contact plant, and these hinder conveying of the process acid and have to be eliminated and disposed of with considerable cost and complexity.

It is also known that hydroxyisobutyric acid can be prepared proceeding from acetone cyanohydrin (ACH) by hydrolysis of the nitrile function in the presence of mineral acids. The prior art describes processes in which ACH is amidated and hydrolysed in the presence of water, wherein the hydroxyl function in the molecular complex is conserved at least in the first steps of the reaction; for example WO 2005/077878, JP H04 193845 A. JP S57 131736. For example, Japanese patent application JP S6361932B2 states that ACH is hydrolysed in a two-stage process to hydroxyisobutyric acid, wherein ACH is first converted in the presence of 0.2 to 1.0 mol of water and 0.5 to 2 equivalents of sulfuric acid, forming the corresponding amide salts. These proposals for an alternative amidation in the presence of water, according to whether it is performed in the presence of methanol or without methanol, lead either to formation of methyl hydroxyisobutyrate (MHIB) or to formation of 2-hydroxyisobutyric acid (HIBAc). For example, according to JP S57 131736, the reaction of ACH with 0.8 to 1.25 equivalents of sulfuric acid is effected in the presence of less than 0.8 equivalent of water below 60° C., and then the reaction with more than 1.2 equivalents of methanol to give MHIB at temperatures of greater than 55° C.

The process for preparing MA is performed essentially analogously to the preparation of MMA. In the preparation of MA and the preparation of MMA, amidation and conversion are often essentially similar or even identical since the particular aim is a maximum MAA yield from ACH. If the MAA-containing third reaction mixture without alcohol is reacted solely with water, the reaction typically leads to crude MAA mixtures as the fourth reaction mixture, whereas, in the presence of water and alcohol (e.g. methanol), the reaction leads to alkyl methacrylates (e.g. MMA) in a fourth reaction mixture. It is known to the person skilled in the art in this connection that, for the selective preparation of MA, it is necessary to provide higher molar proportions of water than is the case for the preparation of MMA.

None of the prior art processes describes a means of monitoring and reducing the level of troublesome by-products, especially of methacrylonitrile (MAN) and acetone, which impair the properties of the alkyl methacrylate and of the alkyl methacrylate polymers to a particular degree.

### OBJECT OF THE INVENTION

It is an object of the invention to overcome the abovementioned disadvantages and to provide an improved process for preparing methacrylic acid and/or alkyl methacrylates based on the ACH-sulfo process, in which the amount of troublesome by-products, especially of methacrylonitrile (MAN) and acetone, can be reduced. This shall improve the product quality of the alkyl methacrylate and of the polymers and shaped bodies produced therefrom. In particular, there shall be an improvement in the processability and the mechanical and optical properties of the alkyl methacrylate polymers.

Moreover, compared to known processes, a comparable or elevated yield of alkyl methacrylate is to be obtained.

### Achievement of the Object

The object was especially achieved in that, in the process according to the invention, the formation of the by-products mentioned is minimized by an optimized reaction regime in the alkyl methacrylate synthesis.

It has been found that, surprisingly, the abovementioned objects are achieved by the process according to the invention. More particularly, it has been found that the amount of troublesome by-products, especially methacrylonitrile (MAN), acetone, methyl propionate and/or methyl isobutyrate, can be reduced when sulfuric acid containing no free SO₃, especially containing small proportions of free water, is used in the conversion of acetone cyanohydrin (amidation and conversion). It has been found to be particularly advantageous to use a sulfuric acid having a concentration in the range from 98.0% by weight to 100.0% by weight, preferably 99.0% by weight to 99.9% by weight. A sulfuric acid having a concentration of 98.0% by weight, for example, is especially understood to mean a sulfuric acid reactant or a sulfuric acid feed stream consisting of sulfuric acid to an extent of 98.0% by weight, based on the overall sulfuric acid reactant or sulfuric acid feed stream. More particularly, the sulfuric acid reactant or sulfuric acid feed stream consists of sulfuric acid and water. A further aspect that makes a crucial contribution to the solution of the problems described is the monitoring and adjustment of the water contents in the acetone feedstock and in the acetone cyanohydrin intermediate. Generally speaking, the process according to the invention permits controlled production and monitoring of the quality and composition of the raw materials and intermediates.

In addition, the troublesome by-products, especially methacrylonitrile (MAN) and acetone, can be effectively discharged from the process to the degree required via an optimized workup of the reaction mixture after the esterification, comprising a suitable discharge and optimized circulation of process streams. More particularly, it has been found that an effective discharge of methacrylonitrile (MAN) and acetone can be achieved in that, in at least one azeotrope distillation step, the by-products are obtained at least partly in the tops fraction as a water-containing heteroazeotrope and are at least partly discharged from the process thereby, optionally after further separation steps. The heteroazeotrope comprising the troublesome by-products may optionally be separated into an aqueous phase and an organic phase, in which case the aqueous phase and/or the organic phase may be at least partly discharged from the process. It is possible here to remove the troublesome by-products from the process together with those streams of matter in which enrichment of the troublesome by-products is not to be expected on account of their physicochemical properties (especially water solubility and volatility). In addition, it is possible by combination of multiple distillation and extraction steps to discharge the troublesome by-products, especially acetone and methacrylonitrile, from the process as derivatives (e.g. acetone in sulfonated form), or to convert them to the target product (e.g. MAN via MAA to MMA).

More particularly, it is possible with the aid of the process according to the invention to perform the industrial ACH-sulfo process more robustly, with lower propensity to faults and with higher yields, with the removal of alkyl methacrylates in the required quality being possible in an effective manner.

### DESCRIPTION OF THE INVENTION

The present invention relates to a process for preparing alkyl methacrylate, preferably methyl methacrylate, comprising
a. reacting acetone and hydrogen cyanide in the presence of a basic catalyst in a first reaction stage to obtain a first reaction mixture comprising acetone cyanohydrin (ACH);
b. working up the first reaction mixture comprising acetone cyanohydrin (ACH);
c. reacting acetone cyanohydrin and sulfuric acid in one or more reactors I in a second reaction stage (amidation) at an amidation temperature in the range from 85° C. to 130° C. to obtain a second reaction mixture comprising sulfoxyisobutyramide and methacrylamide;
d. converting the second reaction mixture, comprising heating to a conversion temperature in the range from 130° C. to 200° C. in one or more reactors II in a third reaction stage (conversion) to obtain a third reaction mixture comprising predominantly methacrylamide (MAA) and sulfuric acid;
e. reacting the third reaction mixture with water and optionally alcohol, preferably water and optionally methanol, in one or more reactors III in a fourth reaction stage (hydrolysis or esterification) to obtain a fourth reaction mixture comprising methacrylic acid and/or alkyl methacrylate, preferably methyl methacrylate;
f. optionally separating alkyl methacrylate from the fourth reaction mixture obtained from the fourth reaction stage;
   wherein the sulfuric acid used in the second reaction stage has a concentration in the range from 98.0% by weight to 100.0% by weight, wherein a pure ACH mixture which is used in the second reaction stage has a water content within a range from 0.1 mol % to 10 mol %, especially 0.4 mol % to 5 mol %, based on the ACH present in the pure ACH mixture, and wherein a total amount of water used in the second reaction stage is within a range from 0.1 mol % to 20 mol %, especially 0.4 mol % to 10 mol %, based on the ACH present in the pure ACH mixture. More particularly, the total amount of water is the sum total of water which is fed in with the pure ACH mixture and any fed in with the sulfuric acid for the second reaction stage.

Preferably, the fourth reaction mixture comprising alkyl methacrylate which is obtained in the fourth reaction stage is worked up in further steps comprising at least one distillation step and/or at least one extraction step. Further preferably, the fourth reaction mixture comprising alkyl methacrylate which is obtained in the fourth reaction stage is guided in gaseous form into a distillation step, wherein a tops fraction comprising alkyl methacrylate, water and alcohol, and a bottoms fraction comprising higher-boiling components are obtained, and wherein the bottoms fraction is recycled fully or partly into the fourth reaction stage. The tops fraction comprising alkyl methacrylate, water and alcohol is preferably separated in a phase separation step or after an extraction step into an organic phase comprising the predominant portion of the alkyl methacrylate and an aqueous phase comprising alcohol and further water-soluble compounds, wherein the aqueous phase is recycled fully or partly into the fourth reaction stage, and the organic phase comprising the predominant portion of the alkyl methacrylate is optionally subjected to an extraction using water as extractant. In addition, the aqueous phase from this extraction is preferably recycled into the fourth reaction stage.

In the context of the present invention, the expression “ppm” without further qualifiers means ppm by weight (e.g. mg/kg).

The expression “stream, phase or fraction comprising a reactant, product and/or by-product” is understood in the context of the invention to mean that the compound(s) mentioned is/are present in the respective stream; for example, the predominant proportion of the reactant, product and/or by-product is to be found in the corresponding stream. In principle, further constituents may be present as well as the compounds mentioned. The naming of the constituents often serves to illustrate respective process step.

The expression “vapour” or “vapour stream” in the context of the invention refers to a gaseous process stream, for example a gaseous top stream from a distillation column. A gaseous vapour stream is typically liquefied by condensation and cooling. In the case of a heteroazeotropic vapour, this mixture typically divides into two phases: a predominantly organic phase and an aqueous, often methanolic phase.

First Reaction Stage (Synthesis of ACH)

The process according to the invention comprises, as step a., the reaction of acetone and hydrogen cyanide in the presence of a basic catalyst in a first reaction stage to obtain a first reaction mixture comprising acetone cyanohydrin (ACH).

Preferably, an acetone reactant which is used in the first reaction stage contains 0.1% by weight to 1% by weight, especially 0.1% by weight to 0.5% by weight, of water, based on the overall acetone reactants. Further preferably, the acetone reactant contains 99% by weight to 99.9% by weight, especially 99.5% by weight to 99.9% by weight, of acetone, based on the overall acetone reactants.

In addition, a hydrogen cyanide reactant which is used in the first reaction stage preferably contains 0.01% by weight to 0.1% by weight of water, based on the overall hydrogen cyanide reactants. Further preferably, the hydrogen cyanide reactant contains at least 99.9% by weight of hydrogen cyanide, based on the overall hydrogen cyanide reactants. A relatively high water content in the hydrogen cyanide reactant in the context of hydrogen cyanide preparation, in a distillative hydrogen cyanide purification in which water is also removed, can have the effect that nitriles that occur as by-product cannot be selectively discharged and hence likewise get into the hydrogen cyanide reactant. A relatively low water content is likewise disadvantageous since, in the distillative hydrogen cyanide purification, the nitriles mentioned remain in the distillation column and can polymerize therein, which leads to blockage.

The acetone cyanohydrin (ACH) can be prepared, for example, as described in Ullmanns Enzyklopädie der technischen Chemie [Ullmann's Encyclopedia of Industrial Chemistry], 4th edition, volume 7. The basic catalyst is preferably an amine, and the reaction of acetone and hydrogen cyanide to give ACH is an exothermic reaction. Such a process stage is described in detail, for example, in DE 10 2006 058 250 and DE 10 2006 059 511. There have been descriptions of alternative processes that use alkali metal hydroxide or other alkali metal derivatives as catalyst in place of the organic amines. Although alkali metal compounds effectively catalyse ACH formation, they can lead to problems, for example deposits, in the conversion reactions, especially sulfuric acid regeneration.

In order to counteract breakdown of the acetone cyanohydrin formed in the first reaction stage, the heat of reaction is typically removed by means of a suitable apparatus. The first reaction stage can be run as a batchwise process or as a continuous process; if a continuous mode of operation is preferred, the reaction is frequently conducted in a loop reactor set up accordingly.

A main feature of a mode of operation that leads to the desired product in high yields is often that, when the reaction time is sufficient, the reaction product is cooled and the reaction equilibrium is moved in the direction of the reaction product. Furthermore, the reaction product of the reaction of acetone and hydrogen cyanide, which is referred to as first reaction mixture, is admixed with a stabilizer to benefit the overall yield, in order to avoid breakdown in the later workup.

The acetone and hydrogen cyanide co-reactants can in principle be mixed in essentially any manner. The method of mixing depends especially on whether a batchwise mode of operation, for example in a batchwise reactor, or a continuous mode of operation, for example in a loop reactor, is chosen.

In principle, it may be advantageous when the acetone is fed into the reaction via at least one reservoir vessel that preferably has a scrubbing tower. Vent conduits that lead off acetone- and hydrogen cyanide-containing output air can thus be guided, for example, through this reservoir vessel. In the scrubbing tower connected to the reservoir vessel, the output air escaping from the reservoir vessel can be scrubbed with acetone, which removes hydrogen cyanide from the output air and returns it to the process. For this purpose, for example, an amount of acetone introduced into the reaction from the reservoir vessel is guided in the substream into the top of the scrubbing tower via a cooler, preferably via a brine cooler, and hence the desired result is achieved.

The temperature of the acetone in the reservoir vessel may in principle be within essentially any range, provided that the acetone is in the liquid state at the appropriate temperature. Advantageously, however, the temperature in the reservoir vessel is about 0 to about 20° C.

In the scrubbing tower, the acetone used for scrubbing may be cooled to a temperature of about 0 to about 10° C. by means of an appropriate cooler, for example by means of a plate cooler with brine. The temperature of the acetone on entry into the scrubbing tower is therefore preferably, for example, about 2 to about 6° C.

The hydrogen cyanide required in the first reaction stage can be introduced into the reactor either in liquid or in gaseous form. It may, for example, be crude gas from the BMA process or from the Andrussow process.

The hydrogen cyanide may be liquefied, for example, for example by the use of an appropriate cooling brine. Rather than liquefied hydrogen cyanide, it is possible to use coking plant gas. For example, hydrogen cyanide-containing coking plant gases, after scrubbing with potash, are scrubbed continuously in countercurrent with acetone, and the reaction to give acetone cyanohydrin can be conducted in the presence of a basic catalyst in two gas scrubbing columns connected in series.

In a further embodiment, it is possible to react a gas mixture containing hydrogen cyanide and inert gases, especially a crude gas from the BMA process or from the Andrussow process, with acetone in the presence of a basic catalyst and acetone cyanohydrin in a gas-liquid reactor.

Preference is given to using a crude BMA gas or a crude Andrussow gas in the first reaction stage. The gas mixture resulting from the abovementioned customary processes for preparing hydrogen cyanide can be used as such or after acid scrubbing. The crude gas from the BMA process in which essentially hydrogen cyanide and hydrogen are formed from methane and ammonia typically contains 22.9% by volume of HCN, 71.8% by volume of H₂, 2.5% by volume of NH₃, 1.1% by volume of N₂, 1.7% by volume of CH₄. In the known Andrussow process, hydrogen cyanide and water are formed from methane, ammonia and atmospheric oxygen. The crude gas from the Andrussow process, when oxygen is used as oxygen source, contains typically 8% by volume of HCN, 22% by volume of H₂O, 46.5% by volume of N₂, 15% by volume of H₂, 5% by volume of CO, 2.5% by volume of NH₃, and 0.5% by volume each of CH₄ and CO₂.

When a non-acid-scrubbed crude gas from the BMA or Andrussow process is used, the ammonia present in the crude gas frequently acts as catalyst for the reaction. Since the ammonia present in the crude gas frequently exceeds the amount required as catalyst and can therefore lead to losses of sulfuric acid used for stabilization, such a crude gas can often be subjected to acid scrubbing in order to eliminate the ammonia therefrom. When such an acid-scrubbed crude gas is used, preference is given to adding a basic catalyst to the reactor for the first reaction stage in a catalytic amount. In principle, known inorganic or organic basic compounds can function here as catalyst.

Hydrogen cyanide in gaseous or liquid form or a hydrogen cyanide-containing gas mixture and acetone are supplied constantly, especially to a loop reactor, in the continuous mode of operation. The loop reactor preferably comprises at least one means of supplying acetone or two or more such means, at least one means of supplying liquid or gaseous hydrogen cyanide, or two or more such means, and at least one means of supplying the catalyst.

Suitable catalysts are in principle any alkaline compounds such as ammonia, sodium hydroxide solution or potassium hydroxide solution, which can catalyse the reaction of acetone and hydrogen cyanide to give acetone cyanohydrin. However, it has been found to be advantageous when the catalyst used is an organic catalyst, especially an amine. Suitable examples are secondary or tertiary amines, such as diethylamine, dipropylamine, triethylamine, tri-n-propylamine and the like.

A loop reactor usable in the first reaction stage preferably has at least one pump and at least one mixing apparatus. Suitable pumps are in principle all pumps suitable for ensuring the circulation of the first reaction mixture in the loop reactor. Suitable mixing apparatuses are both mixing apparatuses having moving elements and what are called static mixers in which immobile baffles are provided. In the case of use of static mixers, suitable examples are those that permit an excess operating pressure of at least 10 bar, for example at least 15 bar or at least 20 bar, under operating conditions without significant restrictions of functioning capacity. Corresponding mixers may consist of plastic or metal. Examples of suitable plastics include PVC, PP; HDPE, PVDF, PFA or PTFE. Metal mixers may consist, for example, of nickel alloys, zirconium, titanium and the like. Likewise suitable, for example, are rectangular mixers.

The catalyst is added in the first reaction stage, preferably within the loop reactor downstream of the pump and upstream of a mixing element provided in the loop reactor. Catalysts are used, for example, in such an amount that the overall reaction in the first reaction stage is run at a pH of not more than 8, especially not more than 7.5 or 7. It is preferable that the pH in the reaction in the first reaction stage is within a range from 6.5 to about 7.5, for example 6.8 to 7.2.

As an alternative to the addition of the catalyst to loop reactor downstream of the pump and upstream of a mixing apparatus, it is also possible in the first reaction stage to feed the catalyst into the loop reactor together with the acetone. In such a case, it may be advantageous when appropriate mixing of acetone and catalyst is ensured before they are fed into the loop reactor. Appropriate mixing can be effected, for example, by the use of a mixer having moving parts or by using a static mixer. This is effected essentially with observation of temperatures and delay times in order to suppress and to avoid unwanted consumption reactions of the catalyst (for example the Cannizzaro reaction) or condensation reactions of the acetone. In order to ensure this, minimum delay times and low temperatures are employed.

If a continuous mode of operation is chosen as the operation method in a loop reactor in the first reaction stage, it may be appropriate to analyse the state of the first reaction mixture by instantaneous or continual analyses. This offers the advantage that it is also possible to react quickly to any changes in state in the first reaction mixture. Furthermore, it is thus possible, for example, to meter in the co-reactants with maximum accuracy in order to minimize yield losses.

Appropriate analysis can be effected, for example, by sampling in the reactor loop. Suitable analysis methods are, for example, pH measurement, measurement of exothermicity or measurement of the composition of the first reaction mixture by suitable spectroscopic methods.

Especially for the purposes of conversion monitoring, quality aspects and safety, it has been found to be useful to determine the conversion in the first reaction mixture via the heat removed from the first reaction mixture and to compare it to the heat released theoretically.

The actual reaction in the first reaction stage, given suitable choice of loop reactor, can in principle be effected in the pipe systems disposed within the loop reactor. But since the reaction is exothermic, in order to avoid yield loss, sufficient cooling or sufficient removal of the heat of reaction should be ensured. It has frequently been found to be advantageous when the first reaction stage is executed in a heat exchanger, preferably in a shell-and-tube heat exchanger. According to the amount to be produced, a different capacity of a corresponding heat exchanger may be chosen.

The shell-and-tube heat exchangers used with preference may be heat exchangers having a bundle of tubes through which liquid flows within a shell through which liquid flows. According to the pipe diameter, packing density etc., it is possible to adjust the heat transfer between the two liquids correspondingly. It is possible in principle in the first reaction stage to conduct the reaction in such a way that the first reaction mixture is run through the heat exchanger within the bundle of tubes itself, and the reaction takes place within the bundle of tubes, with removal of the heat from the bundle of tubes into the shell liquid.

The first reaction mixture can alternatively be conducted through the shell of the heat exchanger, while the liquid used for cooling circulates within the bundle of tubes. In many cases, it has been found here to be advantageous when the first reaction mixture in the shell, for achievement of a higher delay time and better mixing, is distributed by means of baffles, preferably deflecting plates.

The ratio of shell volume to the volume of the bundle of tubes may, for example, according to the design of the reactor, be 10:1 to 1:10; the volume of the shell is preferably greater than the volume of the bundle of tubes (based on the contents of the tubes).

The removal of heat from the reactor in the first reaction stage is preferably adjusted with a coolant, for example with water, in such a way that the reaction temperature (synthesis temperature) in the first reaction stage is within a range from 25 to 45° C., further preferably from 30 to 38° C., especially from 33 to 35° C.

A product, especially the first reaction mixture, is preferably conducted continuously out of the loop reactor for the first reaction stage. The first reaction mixture preferably has a temperature in the region of the abovementioned synthesis temperature, for example a temperature of about 35° C. The product, especially the first reaction mixture, is preferably cooled by means of one or more heat exchangers, especially by means of one or more plate heat exchangers. For example, brine cooling is used here. The temperature of the product, especially of the first reaction mixture, after cooling is preferably 0 to 10° C., especially 1 to 5° C. The product, especially the first reaction mixture, is preferably transferred into a storage vessel having a buffer function. In addition, the product, especially the first reaction mixture, in the storage vessel can be cooled further or kept at a suitable storage temperature, for example by constant removal of a substream from the storage vessel to a suitable heat exchanger, for example a plate heat exchanger. It is entirely possible that further reaction can take place in the storage vessel. Typically, according to the temperature and delay time in the tank, further reaction takes place according to the thermodynamic position of the ACH equilibrium with the reactants, since the catalyst is still in active form. If this reaction is to be suppressed, the catalyst has to be neutralized.

The recycling of the product, especially of the first reaction mixture, into the storage vessel can in principle be effected in any desired manner. However, it has been found to be advantageous in some cases that the product, especially the first reaction mixture, is recycled into the storage vessel via a system composed of one or more nozzles in such a way that corresponding mixing of the stored product, especially of the first reaction mixture, takes place within the storage vessel.

Product, especially first reaction mixture, is further preferably removed continuously from the storage vessel into a stabilization vessel. The product, especially the first reaction mixture, is admixed therein with a suitable acid, for example with H₂SO₄. This deactivates the catalyst and adjusts the reaction mixture to a pH of about 1 to about 3, especially about 2. A suitable acid is especially sulfuric acid, especially sulfuric acid having a content of about 10% to about 105%, especially about 80% to about 98% H₂SO₄. This is important in several aspects with regard to the process according to the invention, especially since the stabilizer acid is also a reactant in the subsequent amidation, and also catalyst for the esterification and hydrolysis. Secondly, the sulfuric acid concentration influences the water management of the overall process and also the water content in the ACH.

The stabilized product, especially the stabilized first reaction mixture, is withdrawn from the first reaction stage, especially the stabilization vessel, and transferred to the workup stage. It is possible here to return a portion of the stabilized product withdrawn, especially of the first reaction mixture, to the stabilization vessel, for example in such a way as to assure sufficient mixing of the vessel via a system composed of one or more nozzles.

Workup of the ACH Synthesized

As step b., the process according to the invention comprises the workup of the first reaction mixture comprising acetone cyanohydrin (ACH). The workup of the first reaction mixture preferably comprises a distillation step in step b., wherein acetone cyanohydrin is at least partly separated from impurities and/or unconverted starting materials and/or by-products that are lower-boiling than acetone cyanohydrin.

In the course of the workup, the first reaction mixture which has been obtained in the upstream first reaction stage is preferably sent to a distillative workup. This frees the first reaction mixture, especially the stabilized first reaction mixture, of low-boiling constituents by means of an appropriate column. A suitable distillation process may be conducted, for example, by means of just one column. However, it is likewise possible, in the context of the workup of the first reaction mixture, to use a combination of two or more distillation columns, also combined with a falling-film evaporator. In addition, it is possible to combine two or more falling-film evaporators or else two or more distillation columns with one another.

The first reaction mixture preferably goes from the storage to the distillation at a temperature within a range from 0 to 15° C., for example from 5 to 10° C. The first reaction mixture can be introduced directly into the column. It has been found to be useful in some cases if the first reaction mixture at low temperature first accepts a portion of the heat from the first reaction mixture that has already been purified by distillation, which is also referred to as pure ACH mixture, via a heat exchanger. This is achieved by heat exchange between the column bottoms and the feed mixture via appropriate apparatuses. Therefore, in the context of a further embodiment, the first reaction mixture is heated to a temperature within a range from 60 to 80° C. by means of a heat exchanger.

The distillative purification is especially effected by means of a distillation column, preferably having more than 10 trays, or by means of a cascade of two or more correspondingly suitable distillation columns. The column bottom is preferably heated with steam. It has been found to be advantageous when the bottom temperature does not exceed a temperature of 140° C.; good yields and good purification are achievable when the bottom temperature is not greater than about 130° C. or not higher than about 110° C. The temperature figures are based on the wall temperature of the column bottom.

The first reaction mixture is preferably supplied to the column body in the upper third of the column. The distillation is preferably conducted at reduced pressure, for example at a pressure of 50 to 900 mbar, especially of 50 to 250 mbar, and with good results between 50 and 150 mbar.

At the top of the column, preferably gaseous reactants (HCN and acetone and traces of water) and impurities, especially acetone and hydrogen cyanide, are removed; the gaseous substances separated off are cooled by means of a heat exchanger or a cascade of two or more heat exchangers. Preference is given here to using brine cooling with a temperature of 0 to 10° C. This at least partially condenses the gaseous constituents of the vapours. The first condensation stage can take place, for example, at standard pressure. However, it is likewise possible and has been found to be advantageous in some cases when this first condensation stage is effected under reduced pressure, preferably at the pressure that exists in the distillation. The condensate is passed onward into a cooled collecting vessel, where it is collected at a temperature of 0 to 15° C., especially at 5 to 10° C.

The gaseous compounds that are not condensable in the first condensation step are preferably removed from the space under reduced pressure by means of a vacuum pump. In principle, any vacuum pump is usable here. However, it has been found to be advantageous in many cases when a vacuum pump that does not lead to input of liquid impurities into the gas stream on account of its design is used here. Preference is therefore given here to using dry-running vacuum pumps, for example.

A gas stream that escapes on the pressure side of the pump is preferably guided through a further heat exchanger which is preferably kept at a temperature of 0 to 15° C. with brine. There is likewise condensation here of constituents in the collecting vessel that already collects the condensates obtained under vacuum conditions. The condensation conducted on the pressure side of the vacuum pump can be effected, for example, by means of a heat exchanger, but also with a cascade of two or more heat exchangers arranged in series and/or in parallel. Gaseous substances that remain after this condensation step are removed and sent to any further utilization, for example a thermal utilization.

The collected condensates can likewise be utilized further. However, it has been found to be advantageous from an economic point of view to return the condensates to the reaction for preparation of acetone cyanohydrin. This is preferably effected at one or more sites that enable access to the loop reactor for the first reaction stage. The condensates may in principle have any composition, provided that they do not disrupt the preparation of the acetone cyanohydrin. In many cases, however, the predominant amount of the condensate will consist of acetone and hydrogen cyanide, for example in a molar ratio of 2:1 to 1:2, frequently in a ratio of about 1:1.

If traces of amine are also condensed in the top of the column, there will be formation of ACH in the liquid phase in the region of the condenser as a result of the presence of the HCN and acetone reactants in liquid form. This tops condensate can either be recycled into the reaction or recycled partly as reflux to the column.

The pure ACH mixture obtained from the bottom of the distillation column is preferably cooled to a temperature of 40 to 80° C. by the cold first reaction mixture supplied by means of a first heat exchanger. Subsequently, the pure ACH mixture is preferably cooled to a temperature of 30 to 35° C. by means of at least one further heat exchanger, and optionally stored intermediately.

Second Reaction Stage (Amidation)

The process according to the invention thus comprises, as step c., the reaction of acetone cyanohydrin and sulfuric acid in one or more reactors I in a second reaction stage (amidation) at an amidation temperature in the range from 85° C. to 130° C. to obtain a second reaction mixture comprising sulfoxyisobutyramide and methacrylamide.

According to the invention, the sulfuric acid used in the second reaction stage has a concentration in the range from 98.0% by weight to 100.0% by weight, preferably of 99.0% by weight to 99.9% by weight, preferably of 99.3% by weight to 99.9% by weight, especially preferably of 99.3% to 99.8% by weight. The use of a sulfuric acid having a zero content of free SO₃, especially a sulfuric acid with a water content of 0.1% to 0.7% by weight, has been found to be particularly advantageous. More particularly, it was thus possible to increase the amidation yield and reduce the proportion of by-products, especially MAN and acetone.

Typically, the reaction (amidation) of acetone cyanohydrin (ACH) and sulfuric acid forms, as main products, alpha-hydroxyisobutyramide (HIBAm) or its hydrogensulfate (HIBAm·H₂SO₄), sulfuric esters of alpha-hydroxyisobutyramide (sulfoxyisobutyramide (SIBA)) or its hydrogensulfate (SIBA·H₂SO₄) and methacrylamide hydrogensulfate (MAA·H₂SO₄), as a solution in excess sulfuric acid. As a result of the presence of small amounts of water defined according to the invention, certain concentrations of MA and HIBAc are likewise detected, to a minor extent in the amidation itself, and in higher concentrations in the subsequent conversion step.

Preferably, the pure ACH mixture which is fed to the second reaction stage is obtained in the workup in step b. More particularly, the pure ACH mixture, proceeding from the workup in step b., is sent to the second reaction stage in unchanged form. Further preferably, the total amount of ACH which is fed to the second reaction stage is fed in with the pure ACH mixture. The pure ACH mixture preferably has an acetone content of not more than 9000 ppm, further preferably of not more than 4000 ppm, more preferably of not more than 1000 ppm, based on the total amount of ACH which is sent to the second reaction stage. Preferably, the pure ACH mixture has an ACH content of not less than 98% by weight, more preferably not less than 98.5% by weight, especially preferably not less than 99% by weight, based on the overall pure ACH mixture. Typically, the pure ACH mixture used in the second reaction stage, especially stream (8a) or (8b) and (8c), contains 98.0% to 99.8% by weight, preferably 98.3% to 99.5% by weight, of acetone cyanohydrin; optionally 0.1% to 1.5% by weight, preferably 0.2% to 1% by weight, of acetone, and water, based on the overall pure ACH mixture, where the sum total is 100% by weight. In addition, the pure ACH mixture may consist of ACH and water. Furthermore, the basic catalyst is present, optionally in neutralized form. This is the case especially when the ACH reaction mixture is obtained as bottom product in the purification.

The second reaction stage is preferably conducted with an excess of sulfuric acid. The sulfuric acid preferably serves as solvent, reactant and catalyst. The sulfuric acid excess can especially serve to keep the viscosity of the second reaction mixture low, which can assure faster removal of heat of reaction and a lower temperature of the second reaction mixture. This can especially bring distinct yield benefits.

Preference is given to using sulfuric acid and acetone cyanohydrin (ACH) in the second reaction stage in a molar ratio of sulfuric acid to ACH in the range from 1.2 to 2.0, further preferably 1.2 to 1.5. The second reaction stage preferably comprises at least two separate reactors I, in which case sulfuric acid and acetone cyanohydrin (ACH) are used in a first reactor I in a molar ratio of sulfuric acid to ACH in the range from 1.6 to 3; preferably 1.7 to 2.6; more preferably 1.8 to 2.3, based on ACH used in the first reactor 1; and wherein sulfuric acid and acetone cyanohydrin (ACH) are used in a last reactor I (for example in a second reactor 1) in a molar ratio of sulfuric acid to ACH in the range from 1.2 to 2.0; preferably from 1.2 to 1.5, based on a total amount of ACH fed to the second reaction stage.

The reaction of acetone cyanohydrin with sulfuric acid in the second reaction stage is exothermic. It is therefore advantageous to largely or at least partly remove the heat of reaction obtained, for example with the aid of suitable heat exchangers, in order to obtain an improved yield. Since the viscosity of the second reaction mixture rises significantly with falling temperature, and hence circulation, flow and heat exchange in the reactors I are limited, excessive cooling should be avoided, however. Furthermore, there can be partial or complete crystallization of ingredients on the heat exchangers at low temperatures in the second reaction mixture, which can lead to abrasion, for example in the pump housings, pipelines and heat exchanger tubes of the reactors I.

For cooling of the reactor circuits, it is possible in principle to use known and suitable cooling media. It is advantageous to use cooling water. Typically, the cooling medium, especially the cooling water, has a temperature below the process conditions chosen. Advantageously, the cooling medium, especially the cooling water, has a temperature in the range from 20 to 90° C., preferably from 50 to 90° C. and more preferably from 60 to 70° C.

To prevent the temperature from going below the crystallization point of methacrylamide and other salts present in the reaction matrix, the heat exchanger (reactor cooler) is typically operated with a secondary hot water circuit. Preference is given here to temperature differences in the inlet/outlet of the apparatus on the product side of about 1 to 20° C., especially 2 to 10° C.

The conversion of acetone cyanohydrin and sulfuric acid in one or more reactors I in the second reaction stage (amidation) is effected at an amidation temperature in the range from 85° C. to 130° C., preferably from 85° C. to 120° C., more preferably from 90° C. to 110° C. The amidation in the second reaction stage in the reactor I or in multiple reactors I is often conducted at standard pressure or moderately reduced pressure.

Typically, the second reaction stage (amidation) can be performed batchwise and/or continuously. The second reaction stage is preferably conducted continuously, for example in one or more loop reactors. Suitable reactors and processes are described, for example, in WO 2013/143812. Advantageously, the second reaction stage can be conducted in a cascade of two or more loop reactors. Especially preferably, the reaction in the second reaction stage is effected in one or more (preferably two) loop reactors.

The first loop reactor is typically operated at a circulation ratio (ratio of circulation volume flow rate to feed volume flow rate) in the range from 5 to 110, preferably 10 to 90, more preferably 10 to 70. In a subsequent loop reactor, the circulation ratio is preferably within a range from 5 to 100, preferably from 10 to 90, more preferably from 10 to 70.

Typically, the statistical delay time in the reactors I, especially in the loop reactors I, is in the range from 5 to 35 minutes, preferably from 8 to 20 minutes.

A suitable loop reactor preferably has the following elements: one or more addition points for ACH, one or more addition points for sulfuric acid, one or more gas separators, one or more heat exchangers, one or more mixers, and a pump. The mixers are frequently executed as static mixers.

The ACH can be added in principle at any point to the one or more reactors I (e.g. loop reactors). However, it has been found to be advantageous when the ACH is added at a well-mixed site. Preference is given to adding the ACH to a mixing element, for example to a mixer having moving parts, or to a static mixer.

The sulfuric acid can be added in principle at any point to the one or more reactors I (e.g. loop reactors). The sulfuric acid is preferably added upstream of the addition of the ACH. Particular preference is given to adding the sulfuric acid on the suction side of the respective reactor pump. It is often possible thereby to improve the pumpability of the gas-containing reaction mixture.

The reactors I (e.g. loop reactors 1) preferably each include at least one gas separator. Typically, it is possible to withdraw product stream (second reaction mixture) continuously via the gas separator on the one hand; on the other hand, it is possible to remove and discharge gaseous by-products. Typically, the gaseous by-product formed is mainly carbon monoxide. Preference is given to guiding a portion of the offgas which is obtained in the amidation into a gas separator together with the third reaction mixture which is obtained in the third reaction stage (conversion).

In a preferred embodiment, the second reaction stage comprises the reaction of acetone cyanohydrin (ACH) and sulfuric acid in at least two separate reaction zones, preferably in at least two loop reactors.

Preference is given to reacting acetone cyanohydrin (ACH) and sulfuric acid in such a way that the reaction volume is divided into at least two reaction zones, and the total amount of ACH is metered separately into the different reaction zones. The amount of ACH which is supplied to the first reactor or to the first reaction zone is preferably not less than the amounts of ACH that are supplied to the downstream reactors or to the downstream reaction zones.

Preference is given to introducing 50-90% by weight, preferably 60% to 75% by weight, of the total volume flow rate of ACH supplied into the first reactor. The remaining amount of ACH supplied is introduced into the second reactor and optionally into further reactors. Typically, the total amount of ACH is divided between the first reactor I and the second reactor I in a mass ratio of first reactor I:second reactor I in the range from 70:30 to 80:20, preferably of about 75:25.

The molar ratio of added sulfuric acid to ACH in the first reactor or in the first reaction zone is greater than the corresponding molar ratio in the downstream reactors or in the downstream reaction zones.

In a particularly preferred embodiment, the conversion in the second reaction stage is effected in two or more loop reactors, in which case the total amount of ACH is metered into the first and at least one further loop reactor. Especially preferably, each loop reactor comprises at least one pump, a heat exchanger cooled with water as medium, a gas separation apparatus, at least one gas conduit connected to the gas separation apparatus, and at least one feed conduit for ACH in liquid form. Preferably, the at least two loop reactors are connected to one another in such a way that the entire resulting reaction mixture from the first loop reactor is guided into the downstream reactors, and the reaction mixture in the downstream reactors is admixed with further liquid ACH and optionally further amounts of sulfuric acid.

The first loop reactor is typically operated at a circulation ratio (ratio of circulation volume flow rate to feed volume flow rate) in the range from 5 to 110, preferably 10 to 90, more preferably 10 to 70. In a subsequent loop reactor, the circulation ratio is preferably within a range from 5 to 100, preferably from 10 to 90, more preferably from 10 to 70.

Typically, after the second reaction stage (amidation), a second reaction mixture is obtained, containing 5% to 25% by weight of sulfoxyisobutyramide (SIBA), 5% to 25% by weight of methacrylamide (MAA) and <3% hydroxyisobutyramide (HIBAm), based in each case on the overall reaction mixture, dissolved in the sulfuric acid reaction matrix.

Third Reaction Stage (Conversion)

The process according to the invention comprises, in step d., converting the second reaction mixture, comprising heating to a conversion temperature in the range from 130° C. to 200° C., preferably 130 to 190° C., more preferably 130 to 170° C., especially 140 to 170° C., in one or more reactors II in a third reaction stage (conversion) to obtain a third reaction mixture comprising predominantly methacrylamide (MAA) and sulfuric acid.

Typically, when heating the second reaction mixture (conversion), which is a sulfuric acid solution comprising SIBA, HIBAm and MAA, each predominantly in the form of the hydrogensulfates, to the conversion temperature in the range from 130 to 200° C., preferably 130 to 180° C., the amount of MAA or MAA H₂SO₄ is increased by dehydration of the HIBAm or SIBA.

Especially preferably, the conversion in the third reaction stage is effected at a temperature in the range from 130 to 200° C., preferably from 130 to 180° C., more preferably 140 to 170° C., and a delay time in the range from 2 to 30 minutes, preferably 3 to 20 minutes, especially preferably 5 to 20 minutes. Preference is given to heating in the third reaction stage (conversion) over a minimum period of time. In particular, the heating in the third reaction stage is effected for a period of 1 to 30 minutes, preferably 1 to 20 minutes, 2 to 15 minutes, more preferably 2 to 10 minutes. In a preferred embodiment, the third reaction stage (conversion) comprises the heating of the reaction mixture, for example in one or more preheater segment(s), and the guiding of the reaction mixture under approximately adiabatic conditions, for example in one or more delay segments.

The conversion can be conducted in known reactors that enable the attainment of the temperatures mentioned within the periods of time mentioned. The energy can be supplied here in a known manner, for example by means of steam, electrical energy or electromagnetic radiation, such as microwave radiation. Preference is given to conducting the conversion in the third reaction stage in one or more heat exchangers.

In a preferred embodiment, the conversion in the third reaction stage is conducted in a heat exchanger comprising a two-stage or multistage arrangement of pipe coils. The multistage pipe coils are preferably arranged in opposing rotations.

The heat exchanger may be combined, for example, with one or more gas separators. For example, it is possible to guide the reaction mixture through a gas separator after it has left the first pipe coil of the heat exchanger and/or after it has left the second pipe coil of the heat exchanger. It is especially possible here to separate gaseous by-products from the reaction mixture.

Typically, the second reaction mixture obtained in the second reaction stage is guided completely into reactor II of the third reaction stage.

Preferably, the third reaction mixture which is obtained after the conversion is guided into a gas separator, wherein gaseous by-products can be at least partly separated from the third reaction mixture. Typically, the degassed third reaction mixture is guided fully into the fourth reaction stage (esterification). Preferably, the offgas which is obtained after the conversion in the gas separator is discharged fully or partly from the process. Further preferably, the offgas which is obtained after the conversion in the gas separator is guided fully or partly into the fourth reaction stage (esterification).

More particularly, the process according to the invention enables reduction in the amount of troublesome by-products, preferably in the amounts of MAN, acetone, MA and/or HIBAm, in the third reaction mixture (after amidation and conversion). Preferably, the third reaction mixture contains not more than 3% by weight, preferably not more than 2% by weight, of MA, not more than 2% by weight, preferably not more than 1.5% by weight, more preferably not more than 1% by weight, of HIBAm, and not more than 0.3% by weight, especially not more than 0.03% by weight, of methacrylonitrile (MAN), based in each case on the overall third reaction mixture.

Preferably, the third reaction mixture (after amidation and conversion) contains 30% to 40% by weight of methacrylamide (MAA), based on the overall third reaction mixture. Preferably, the third reaction mixture (after amidation and conversion) contains 30% to 40% by weight of MAA, 0% to 3% by weight of MA and 0.2% to 1.5% by weight, preferably 0.2% to 1% by weight, of HIBAm and 0.001% to 0.3% by weight of MAN, based in each case on the overall third reaction mixture.

Fourth Reaction Stage (Hydrolysis or Esterification)

In one embodiment, the process according to the invention comprises, in step e., the reaction of the third reaction mixture comprising predominantly methacrylamide with water in one or more reactors III in a fourth reaction stage (hydrolysis) to obtain a fourth reaction mixture comprising methacrylic acid. The word “predominantly” is especially understood to mean that the content of the component mentioned, based on the mixture, is more than 50% by weight.

The hydrolysis can be performed in one or more suitable reactors III, for example in heated tanks or tubular reactors at temperatures in the range from 90 to 130° C., preferably from 100 to 125° C. The third reaction mixture is preferably reacted with an excess of water, with use of a molar ratio of water to methacrylamide in the range from 1 to 6, preferably 3 to 6, in the fourth reaction stage.

The hydrolysis with water typically affords a fourth reaction mixture comprising methacrylic acid and possibly hydroxyisobutyric acid (HIBAc) and further above-described by-products, and especially water.

In a further embodiment, the process according to the invention comprises, in step e., the reaction of the third reaction mixture comprising predominantly methacrylamide with alcohol and water, preferably with methanol and water, in one or more reactors III in a fourth reaction stage (esterification) to obtain a fourth reaction mixture comprising alkyl methacrylate.

The conditions for the esterification on an industrial scale are known to the person skilled in the art and are described, for example, in U.S. Pat. No. 5,393,918.

The conversion in the fourth reaction stage (esterification) is preferably conducted in one or more suitable reactors III, for example in heated tanks. In particular, it is possible to use steam-heated tanks. In a preferred embodiment, the esterification is effected in two or more, for example three or four, successive tanks (tank cascade).

Typically, the esterification is conducted at temperatures in the range from 90 to 180° C., preferably from 100 to 150° C., at pressures up to 7 bar, preferably of not more than 2 bar, and using sulfuric acid as catalyst.

Preference is given to reacting the third reaction mixture with an excess of alcohol and water, preferably an excess of methanol and water. The addition of the third reaction mixture comprising predominantly methacrylamide and the addition of alcohol are preferably effected in such a way as to result in a molar ratio of methacrylamide to alcohol in the range from 1:0.7 to 1:1.6. In a preferred embodiment, the reaction in the fourth reaction stage is effected in two or more reactors III, in which case there is a molar ratio of methacrylamide to alcohol in the first reactor III in the range from 1:0.7 to 1:1.4, preferably in the range from 1:0.9 to 1:1.3, and in which case there is a molar ratio of methacrylamide to alcohol in the second and possible downstream reactors III in the range from 1:1.0 to 1:1.3. It should generally be noted that good, efficient and highly selective conversions are achieved especially when MAA in every partial conversion range is provided with methanol and water in a sufficient molar amount. This is true over all reactors, and also for individual reactors.

Preferably, the alcohol supplied to the fourth reaction stage (esterification) is composed of alcohol freshly supplied to the process (fresh alcohol) and of alcohol present in recycled streams (recycling streams) in the process according to the invention. It is additionally possible in the process according to the invention to use alcohol present in recycling streams from downstream processes.

The alcohol may especially be selected from linear, branched, saturated and unsaturated C₁-C₆ alcohols, preferably C₁-C₄ alcohols. More particularly, the alcohol is a saturated C₁-C₄ alcohol. The alcohol is preferably selected from methanol, ethanol, propanol and butanol, or branched isomers of the C3 to C4 alcohols. The alcohol is more preferably methanol.

Typically, water is added to the reactor III or to the reactors III of the fourth reaction stage in such a way that the concentration of water is in the range from 10% to 30% by weight, preferably 15% to 25% by weight, based in each case on the overall reaction mixture in the reactor III.

In the case of the hydrolysis of MAA to MA, when the organic acid is the target product, higher water concentrations are also achieved in the reaction product.

In principle, the water supplied to the fourth reaction stage (esterification) may come from any source and may contain various organic compounds, provided that no compounds are present that have an adverse effect on the esterification or the downstream process stages. The water supplied to the fourth reaction stage preferably comes from recycled streams (recycling streams) in the process according to the invention, for example from the purification of the alkyl methacrylate. It is additionally possible to supply fresh water, especially demineralized water or well water, to the fourth reaction stage (esterification).

Esterification with methanol typically affords a fourth reaction mixture comprising alkyl methacrylate (especially MMA), methyl hydroxyisobutyrate (MHIB) and further above-described by-products, and also significant amounts of water and unconverted alcohol (especially methanol).

In a preferred embodiment, the esterification is effected in two or more (especially three or four) successive tanks (tank cascade), wherein the liquid overflow and the gaseous products are guided from the first tank into the second tank. The corresponding procedure is typically followed with possible downstream tanks. More particularly, such a mode of operation can reduce foam formation in the tanks. In the second tank and in the possible downstream tanks, it is likewise possible to add alcohol. The amount of alcohol added here is preferably at least 10% less compared to the preceding tank. The concentration of water in the various tanks may typically be different. The temperature of the third reaction mixture fed into the first tank is typically in the range from 80 to 180° C. The temperature in the first tank is typically in the range from 90 to 180° C., and the temperature in the second and in the possible downstream tanks is in the range from 100 to 150° C., more preferably between 100 and 130° C.

In a preferred embodiment, the third reaction mixture which is obtained in the fourth reaction stage is removed from the reactors III in gaseous form (vapour) and sent to further workup, for example a distillation step. More particularly, the third reaction mixture can be guided in the form of a vapour into the bottom of a downstream distillation column K1 (primary column K1). If a cascade consisting of multiple reactors III, for example multiple stirred tanks, is used, it is possible to remove the resultant reaction mixture as a vapour stream in each tank and guide it to further workup. Preferably, only the reaction mixture formed in the last tank (as the third reaction mixture) is removed as vapour stream and guided to further workup.

This vapour stream formed in the esterification (third reaction mixture) is typically an azeotropic mixture (actually a mixture of pure substances and various azeotropes) comprising water, alkyl methacrylate, alcohol, fractions of MA, and the by-products described, e.g. methacrylonitrile and acetone. Typically, this vapour stream formed in the esterification (third reaction mixture) has a temperature in the range from 60 to 120° C., where the temperature depends on the alcohol used. Typically, this vapour stream formed in the esterification has a temperature in the range from 70 to 90° C. if methanol is used as alcohol.

It is advantageously possible to add one or more stabilizers in various streams of the process according to the invention in order to prevent or reduce polymerization of the methacrylic acid and/or of the alkyl methacrylate. For example, it is possible to add a stabilizer to the third reaction mixture obtained after the hydrolysis or esterification. It is further advantageous to add a stabilizer to the tops fraction from the first distillation step K1 (primary column K1). A combination of various stabilizers and the supply of small amounts of oxygenous gases to the various workup stages has been found to be useful.

A waste stream (e.g. (11)) consisting essentially of dilute sulfuric acid is preferably removed from the fourth reaction stage (esterification). This waste stream is typically discharged from the process. This waste stream, especially together with one or more aqueous waste streams from the process according to the invention, is preferably sent to a process for regeneration of sulfuric acid or a process for obtaining ammonium sulfate.

Workup of the Third Reaction Mixture

The process according to the invention optionally comprises, in step f., the separation of methacrylic acid or alkyl methacrylate from the third reaction mixture.

Methacrylic acid can be separated off in any manner known to the person skilled in the art, and the separation may comprise steps of phase separation, extraction and distillation, for example. An illustrative workup method is set out in DE 10 2008 000 787 A1, but it is also possible to employ other sequences of basic operations for the preparation of methacrylic acid of a desired purity.

The separation (workup) of alkyl methacrylate from the third reaction mixture preferably comprises at least two distillation steps in which the methacrylonitrile (MAN) and acetone by-products are obtained at least partly as a water-containing heteroazeotrope in the tops fraction and are especially at least partly separated from the alkyl methacrylate, wherein the water-containing heteroazeotrope comprising methacrylonitrile (MAN) and acetone is discharged at least partly from the process from at least one of these distillation steps, and wherein at least one stream comprising methacrylonitrile and acetone is at least partly recycled into the fourth reaction stage.

The at least one stream comprising methacrylonitrile and acetone which is at least partly recycled into the fourth reaction stage (esterification) is preferably a water-containing substance mixture comprising methacrylonitrile and acetone from at least one of the distillation steps, as described above.

For example, the aqueous phase and/or the organic phase of the water-containing heteroazeotrope may be discharged from the process from at least one distillation step and/or mixtures thereof, optionally after further workup steps, such as condensation, phase separation, extraction and scrubbing steps.

Preferably, at least one aqueous phase which is obtained by means of condensation and phase separation of the water-containing heteroazeotrope from at least one of the distillation steps is recycled fully or partly, optionally after an extraction step, into the fourth reaction stage (esterification), where it is contacted with the third reaction mixture comprising predominantly methacrylamide and sulfuric acid.

Preferably, at least one aqueous phase which is obtained by means of condensation and phase separation of the water-containing heteroazeotrope from at least one of the distillation steps is discharged fully or partly from the process, optionally after an extraction step.

In a further preferred embodiment, the water-containing heteroazeotrope from at least one of the distillation steps is discharged fully or partly from the process, at least partly in the form of a gaseous stream, optionally after a scrubbing step. For example, the water-containing heteroazeotrope from at least one distillation step can be removed in the form of a vapour stream and discharged from the process in gaseous form (as an offgas stream), optionally after further workup steps, for example selected from condensation, phase separation, extraction and scrubbing steps.

Preferably, the separation of alkyl methacrylate from the fourth reaction mixture (step f) comprises at least one phase separation step in which the aqueous heteroazeotrope from at least one of the distillation steps is separated into an aqueous phase comprising methacrylonitrile and acetone, and an organic phase comprising predominantly alkyl methacrylate, wherein the aqueous phase is partly discharged from the process and/or partly recycled into the fourth reaction stage, and wherein the organic phase comprising predominantly alkyl methacrylate is recycled fully or partly into the at least one distillation step.

As well as the troublesome MAN and acetone by-products, the water-containing heteroazeotrope which is obtained as tops fraction in the at least one distillation step typically comprises alcohol, for example methanol, water and methyl formate.

In general, MAN forms an azeotrope both with methanol and with methyl methacrylate (MMA), which means that the removal of MAN entails a high level of separation complexity. Typically, the troublesome MAN by-product, in the at least one distillation step as described above, is therefore typically obtained both in the tops fraction as water-containing heteroazeotrope and in the bottoms fraction.

Primary Column (K1) and Prepurification

The removal of alkyl methacrylate in step f of the process according to the invention preferably comprises the prepurification of the fourth reaction mixture which is obtained in the esterification. More particularly, the prepurification comprises at least one distillation step K1 (primary column), at least one phase separation step (e.g. phase separator 1) and at least one extraction step (e.g. extraction step). In a further embodiment, the prepurification comprises at least two distillation steps, e.g. primary column K1 and primary stripper column K4, and at least one phase separation step (e.g. phase separator).

Preferably, the fourth reaction mixture obtained in the fourth reaction stage is evaporated continuously, wherein the resultant vapour stream is fed to a first distillation step K1 (primary column K1) in which a tops fraction comprising alkyl methacrylate, water and alcohol, and a bottoms fraction comprising higher-boiling components are obtained, and wherein the bottoms fraction is recycled fully or partly into the fourth reaction stage. More particularly, the tops fraction of the distillation step K1 is a water-containing heteroazeotrope comprising methacrylonitrile and acetone. Water-containing heteroazeotrope in this case refers to a vapour stream which divides into two phases after condensation and liquefaction, or divides into different phases after addition of water.

In a preferred embodiment (variant A), the tops fraction of the distillation step K1 comprising alkyl methacrylate, water and alcohol is separated in a phase separation step (phase separator 1) into an organic phase OP-1 comprising the predominant portion of the alkyl methacrylate and an aqueous phase WP-1 comprising alcohol and further water-soluble compounds, with the aqueous phase typically being recycled fully or partly into the fourth reaction stage. Further preferably, the organic phase OP-1 comprising the predominant portion of the alkyl methacrylate is subjected to an extraction, preferably using water as extractant, wherein the aqueous phase from this extraction is typically recycled fully or partly into the fourth reaction stage (esterification).

In a further preferred embodiment (variant B), the tops fraction from distillation step K1 comprising alkyl methacrylate, water and alcohol is guided as vapour stream into a further distillation step K4 (e.g. primary stripper column (1)), in which a water-containing heteroazeotrope comprising methacrylonitrile and acetone is obtained as tops fraction, and a bottoms fraction comprising alkyl methacrylate. Preferably, the tops fraction from distillation step K4, optionally after a scrubbing step, preferably after a scrubbing step with alcohol (e.g. methanol), is discharged fully or partly from the process in the form of a gaseous stream. The bottoms fraction from distillation step K4 is preferably separated in a phase separation step (phase separator II) into an aqueous phase WP-2 comprising methacrylonitrile and acetone, and an organic phase OP-2 comprising the predominant portion of the alkyl methacrylate. Typically, the aqueous phase WP-2 comprising methacrylonitrile and acetone is recycled fully or partly into the fourth reaction stage (esterification).

Azeotrope Column (K2) and Purifying Column (K3)

The separation of alkyl methacrylate from the fourth reaction mixture (step f) preferably comprises guiding an organic phase (from extraction or from phase separator) comprising a predominant portion of the alkyl methacrylate into a distillation step K2 (azeotrope column) in which the tops fraction obtained is a water-containing heteroazeotrope comprising methacrylonitrile and acetone, and the bottoms fraction obtained is a crude alkyl methacrylate product.

Preference is given to conducting distillation step K2 (azeotrope column) under reduced pressure. Preference is given to preheating the organic feed of distillation step K2 and guiding it to the top of distillation column K2. It is typically possible to heat the top of the column indirectly with low-pressure steam by means of an evaporator.

Preference is given to removing a water-containing heteroazeotrope comprising alkyl methacrylate (e.g. MMA), water, alcohol (especially methanol), acetone, methacrylonitrile and further low boilers at the top of distillation column K2 (azeotrope column).

Typically, a bottoms fraction comprising the predominant proportion of the alkyl methacrylate, especially methyl methacrylate, and virtually free of low boilers, but contaminated with high boilers, for example methacrylic acid (MA) and methyl hydroxyisobutyrate (MHIB), is obtained in distillation step K2 (azeotrope column). The crude alkyl methacrylate product which is obtained as bottoms fraction from distillation step K2 (azeotrope distillation) preferably contains at least 99.0% by weight of alkyl methacrylate. The crude alkyl methacrylate product which is obtained as bottoms fraction from distillation step K2 (azeotrope distillation) preferably has a MAN content of 20 to 2000 ppm.

In a preferred embodiment, the tops fraction from distillation step K2 (azeotrope column) is first guided as vapour stream into a condenser and condensed stepwise under reduced pressure. This stepwise condensation preferably gives rise to a biphasic condensate I in the first stage (on the suction side of the condenser), and a further condensate II in the second stage (on the pressure side of the condenser). The offgas formed in the stepwise condensation (especially in the condensation on the pressure side) is preferably discharged from the process, optionally after a scrubbing step.

In a preferred embodiment (variant A), the biphasic condensate I from the first stage of the condensation is guided into a phase separator, and the further condensate II from the second stage of the condensation is used as extractant in a downstream extraction step.

In another preferred embodiment (variant B), the liquid phases from the stepwise condensation are combined and guided into a phase separator in the form of a liquid biphasic stream.

The water-containing heteroazeotrope which is obtained as tops fraction in distillation step K2, typically after condensation, is preferably separated in a phase separator II into at least one organic phase OP-2 comprising alkyl methacrylate and at least one aqueous phase WP-2 comprising MAN, acetone and methanol. The aqueous phase WP-2 and/or the organic phase OP-2 is preferably discharged fully or partly from the process. More particularly, the aqueous phase WP-2 is recycled fully or partly into the fourth reaction stage (esterification), typically after a phase separation. Especially preferably, the aqueous phase WP-2 comprising methacrylonitrile (MAN) and acetone is partly discharged from the process and partly recycled into the fourth reaction stage (esterification).

The aqueous phase WP-2 often contains 10 to 10 000 ppm of MAN, based on the overall aqueous phase WP-2.

Preferably, the organic phase OP-2 of the water-containing heteroazeotrope which is obtained as tops fraction in distillation step K2 is recycled fully or partly, preferably fully, into distillation step K2, typically after a phase separation. In particular, the organic phase OP-2 comprises alkyl methacrylate and methacrylonitrile (MAN).

Typically, the predominant proportion of MAN present in the tops fraction from distillation step K2 is to be found in the organic phase (OP-2) of the heteroazeotrope. Preferably, the complete or partial recycling of the organic phase of the heteroazeotrope (OP-2) into distillation step K2 can achieve enrichment of the troublesome by-products, especially MAN, and hence more effective removal, for example via the aqueous phase of the heteroazeotrope (WP-2). In a preferred embodiment, the weight ratio of MAN in the aqueous phase WP-2 to MAN in the organic phase OP-2 is greater than 0.01.

In a preferred embodiment, the crude alkyl methacrylate product from distillation step K2 is guided into a further distillation step K3 (purifying column) in which the alkyl methacrylate is separated from higher-boiling compounds, and in which the tops fraction obtained is a pure alkyl methacrylate product. Preferably, the pure alkyl methacrylate product from distillation step K3 contains at least 99.9% by weight, preferably at least 99.95% by weight, based on the pure alkyl methacrylate product, of alkyl methacrylate. Preferably, the pure alkyl methacrylate product from distillation step K3 contains a content of methacrylonitrile (MAN) in the range from 10 to 300 ppm, preferably 10 to 100 ppm, more preferably 10 to 80 ppm, especially preferably 50 to 80 ppm, based on the pure alkyl methacrylate product. The pure alkyl methacrylate product preferably has a content of acetone of not more than 10 ppm, preferably of not more than 2 ppm, more preferably of not more than 1 ppm, based on the pure alkyl methacrylate product.

In a preferred embodiment, in the second distillation step K2 (azeotrope column), the bottoms fraction obtained is a crude alkyl methacrylate product preferably containing at least 99.0% by weight of alkyl methacrylate, wherein the crude alkyl methacrylate product is purified in a further distillation step K3 (purifying column), wherein the tops fraction obtained is a pure alkyl methacrylate product having a content of methacrylonitrile in the range from 10 to 300 ppm, preferably 10 to 100 ppm, more preferably 10 to 80 ppm, especially preferably 50 to 80 ppm, based on the pure alkyl methacrylate product.

The crude alkyl methacrylate product from distillation step K2 is preferably guided into distillation step K3 (purifying column) in liquid boiling form. The feed from distillation step K3 is preferably in the middle of purifying column K3. The energy input into distillation column K3 is typically effected by means of an evaporator heated with low-pressure steam. Distillation step K3 (purifying column), like distillation step K2, is preferably conducted under reduced pressure.

Typically, the distillate stream fully condensed at the top of column K3 is divided into a product stream and a recycle stream into the column. The quality of the pure alkyl methacrylate product can be controlled, for example, via the reflux ratio. The bottom stream is preferably recycled into the esterification.

Typically, the bottoms fraction from distillation step K3 (purifying column) (e.g. (O)) can be recycled fully or partly into the fourth reaction stage (esterification). More particularly, it is possible thereby to recover alkyl methacrylate present.

Variant A

In a preferred embodiment of the invention (also referred to as variant A), the separation of alkyl methacrylate from the fourth reaction mixture comprises
(i) first distilling the fourth reaction mixture obtained in the fourth reaction stage (esterification) in a first distillation step K1 (primary column) to obtain a first water-containing heteroazeotrope comprising methacrylonitrile and acetone as tops fraction;
(ii) separating the first water-containing heteroazeotrope as condensate in a phase separation step (phase separator 1) into an aqueous phase WP-1 and an organic phase OP-1 comprising the predominant portion of the alkyl methacrylate;
(iii) guiding the organic phase OP-1, optionally after an extraction step, into a second distillation step K2 (azeotrope column), wherein the tops fraction obtained is a second water-containing heteroazeotrope comprising methacrylonitrile and acetone;
(iv) separating at least a portion of the second water-containing heteroazeotrope in a phase separation step (phase separator II) into an aqueous phase WP-2 comprising methacrylonitrile and acetone, and an organic phase OP-2,
   wherein the organic phase OP-2 is recycled fully or partly into the second distillation step K2,
   and wherein the organic phase WP-2 comprising methacrylonitrile and acetone is partly recycled into the fourth reaction stage (esterification) and partly discharged from the process, optionally after an extraction step.

In a preferred embodiment (variant A), the aqueous phase WP-1 is recycled fully or partly into the fourth reaction stage (esterification), and the organic phase OP-1 comprising the predominant portion of the alkyl methacrylate is subjected to an extraction using water as extractant, wherein the aqueous phase of this extraction is recycled into the fourth reaction stage and the organic phase of this extraction is guided into the second distillation step K2 (azeotrope column).

Preference is given to adding water, typically demineralized water or well water, in the phase separation step (phase separator II), in which at least a portion of the second water-containing heteroazeotrope is separated into an aqueous phase WP-2 and an organic phase OP-2, which typically improves the phase separation.

In a preferred embodiment (variant A), a portion of the aqueous phase WP-2 comprising methacrylonitrile and acetone is subjected to an extraction to obtain an aqueous phase WP-3 and an organic phase OP-3, wherein the aqueous phase WP-3 is discharged fully or partly from the process, and wherein the organic phase OP-3 is recycled fully or partly into the fourth reaction stage. It is optionally possible to at least partly discharge the organic phase OP-3 from the process. The organic phase OP-3 is preferably discharged from the process as cleavage acid together with the waste acid from the esterification. More particularly, the aqueous phase WP-3, for example together with the waste acid from the esterification, can be sent to a downstream process for regeneration of sulfuric acid or a downstream process for obtaining ammonium sulfate.

Typically, in the above-described variant A, the discharge of troublesome by-products, typically MAN and acetone, is effected via a portion of the aqueous phase WP-2, wherein the loss of alkyl methacrylate can be reduced by a downstream extraction step.

Preferably, the tops fraction from distillation step K2 (second water-containing heteroazeotrope) is first guided as vapour stream into a condenser and condensed stepwise under reduced pressure. What is preferably obtained here is a biphasic condensate I in the first stage of the condensation (on the suction side of the condenser), which is guided into a phase separator. A further condensate II is preferably additionally obtained in the second stage of the condensation (on the pressure side of the condenser), which is used as extractant in the extraction of the aqueous phase WP-2 or a portion of the aqueous phase WP-2.

In a further embodiment, a portion of the aqueous phase WP-2 comprising methacrylonitrile and acetone is subjected to an extraction to obtain an aqueous phase WP-3 and an organic phase OP-3, wherein the aqueous phase WP-3 is subjected to a further distillation step K5, wherein a tops fraction comprising methacrylonitrile is obtained in distillation step K5, which is discharged from the process, and wherein a bottoms fraction comprising water is obtained in distillation step K5, which is recycled fully or partly into the extraction, and wherein the organic phase OP-3 is recycled fully or partly into the fourth reaction stage. Typically, the aqueous bottoms fraction from distillation step K5 is largely free of methacrylonitrile. Typically, with the aid of the further distillation step K5, the discharged wastewater stream can be purified, and the disposal of the waste stream simplified.

Variant B

In a preferred embodiment of the invention (also referred to as variant B), the separation of alkyl methacrylate from the fourth reaction mixture comprises
(i) first distilling the fourth reaction mixture obtained in the fourth reaction stage in a first distillation step K1 (primary column) to obtain a first water-containing heteroazeotrope comprising methacrylonitrile and acetone as tops fraction,
(ii) guiding the first water-containing heteroazeotrope as a vapour stream into a further distillation step K4 (primary stripper) in which a further water-containing heteroazeotrope comprising methacrylonitrile and acetone is obtained as tops fraction, and a bottoms fraction comprising alkyl methacrylate,
(iii) discharging the tops fraction from distillation step K4, optionally after a scrubbing step, fully or partly from the process in the form of a gaseous stream,
(iv) separating the bottoms fraction from distillation step K4 in a phase separation step (phase separator II) into an aqueous phase WP-2 comprising methacrylonitrile and acetone, and an organic phase OP-2, wherein the aqueous phase WP-2 comprising methacrylonitrile and acetone is recycled fully or partly into the fourth reaction stage,
(v) guiding the organic phase WP-2 fully or partly into a second distillation step K2 (azeotrope column) in which the tops fraction obtained is a second water-containing heteroazeotrope comprising methacrylonitrile and acetone, which is condensed fully or partly and guided into the phase separation step (phase separator II) according to (iv).

Typically, in distillation step K4 (primary stripper), the tops fraction obtained is a low-boiling mixture comprising methanol, acetone, methacrylic esters and water, and the tops fraction obtained is an azeotropically boiling mixture comprising alkyl methacrylate and water.

In a preferred embodiment (variant B), the reflux in distillation step K4 (primary stripper) is produced by means of a partial condenser adjusted such that the tops fraction is discharged from column K4 in the form of a vapour and a liquid condensate comprising alkyl methacrylate is returned to the column as reflux. A portion of the reflux from distillation column K4 is preferably removed in the form of a liquid sidestream and guided as reflux into distillation column K1 (primary column).

Typically, the bottoms fraction from distillation step K4 (primary stripper) is an azeotropic mixture comprising alkyl methacrylate, water, small amounts of low boilers (e.g. methanol, acetone) and high boilers (e.g. hydroxyisobutyric esters). The bottoms fraction from distillation step K4 is preferably cooled and separated in a phase separator II, preferably together with a further reflux stream into an organic phase OP-2 and an aqueous phase WP-2. Typically, the aqueous phase WP-2 comprises water, alcohol, acetone and alkyl methacrylate. The aqueous phase WP-2 can preferably be mixed with fresh water, e.g. demineralized water (DM water), and sent to the esterification in the form of a combined reflux stream. Typically, it is possible thereby to cover the water demand of the esterification and recover reactants.

The tops fraction from distillation step K4 is preferably guided as a vapour stream into an offgas scrubbing column, where it is scrubbed with fresh alcohol, e.g. methanol, as scrubbing medium. The scrubbed offgas stream is preferably discharged fully or partly from the process. The organic stream comprising methanol and alkyl methacrylate is preferably obtained in the bottoms from the offgas scrubbing column, and is recycled into the esterification. This organic reflux stream may be distributed here between various esterification reactors.

Further Steps

In a preferred embodiment, the process according to the invention comprises a regeneration of sulfuric acid, wherein a portion of the fourth reaction mixture obtained in the fourth reaction stage and at least one aqueous or organic waste stream comprising sulfuric acid, ammonium hydrogensulfate and sulfonated acetone derivatives that results from the discharge of the water-containing heteroazeotrope comprising methacrylonitrile and acetone is sent to a thermal regeneration step in which sulfuric acid is obtained, which is recycled into the fourth reaction stage.

In a preferred embodiment, the process according to the invention comprises obtaining ammonium sulfate, wherein a portion of the fourth reaction mixture obtained in the fourth reaction stage and at least one aqueous or organic waste stream comprising sulfuric acid, ammonium hydrogensulfate and sulfonated acetone derivatives that results from the discharge of the water-containing heteroazeotrope comprising methacrylonitrile and acetone is sent to a thermal regeneration step in which ammonium sulfate is obtained by means of crystallization, which is separated off as a by-product.

A waste stream consisting essentially of dilute sulfuric acid which is removed from the reactor Ill for esterification and/or one or more aqueous waste streams from the process is preferably sent to a process for regeneration of sulfuric acid or to a process for obtaining ammonium sulfate.

Processes for regeneration of sulfuric acid and processes for obtaining ammonium sulfate from cleavage acid are known to the person skilled in the art and are described, for example, in WO 02/23088 A1 and WO 02/23089 A1. The embedding of processes for regeneration of sulfuric acid into a process for preparing alkyl methacrylates by the ACH-sulfo process is described, for example, in DE 10 2006 059 513 or DE 10 2006 058 250.

### DESCRIPTION OF THE FIGURES

FIG. 1 describes the reaction network of the formation of methacrylic acid and/or methyl methacrylate proceeding from methane and ammonia, and acetone. Proceeding from methane (CH₄) and ammonia (NH₃), it is possible to prepare hydrogen cyanide via the BMA process (hydrogen cyanide from methane and ammonia) by means of catalytic dehydrogenation (CH₄+NH₃→HCN+3 H₂) (variant 1). Alternatively, it is possible to prepare hydrogen cyanide via the Andrussow process proceeding from methane and ammonia, with addition of oxygen (CH₄+NH₃+1.5 O₂→HCN+3 H₂O) (variant 2). In the next step, proceeding from acetone and hydrogen cyanide, acetone cyanohydrin (ACH) is prepared with addition of a basic catalyst (e.g. diethylamine Et₂NH or inorganic bases). The hydroxyl group of acetone cyanohydrin is subsequently esterified with sulfuric acid, initially giving sulfoxyisobutyronitrile (SIBN). The nitrile group of sulfoxyisobutyronitrile (SIBN) can be hydrolysed in the next step under the action of sulfuric acid and water, giving sulfoxyisobutyramide hydrogensulfate (SIBA·H₂SO₄). A side reaction that can proceed is the formation of methacrylonitrile (MAN) with elimination of sulfuric acid from SIBN. Sulfoxyisobutyramide hydrogensulfate (SIBA·H₂SO₄) can additionally be partly hydrolysed to give alpha-hydroxyisobutyramide hydrogensulfate (HIBAm·H₂SO₄). Likewise possible is the reverse reaction to give the sulfuric ester SIBA·H₂SO₄. A by-product formed may be alpha-hydroxyisobutyric acid (HIBAc) via further hydrolysis of HIBAm·H₂SO₄. Proceeding from SIBA·H₂SO₄, with the elimination of sulfuric acid, methacrylamide hydrogensulfate (MAA·H₂SO₄) is formed (conversion). The gradual reaction of HIBAm or HIBAc to give MA or MAA can likewise proceed as an elimination reaction with elimination of NH₄HSO₄ or water. Methacrylamide hydrogensulfate (MAA·H₂SO₄) can subsequently be converted by hydrolysis to methacrylic acid (MA) or by esterification with methanol (MeOH) to methyl methacrylate (MMA). If alpha-hydroxyisobutyric acid (HIBAc) is entrained into the esterification, it can be converted to methyl alpha-hydroxyisobutyrate (MHIB).

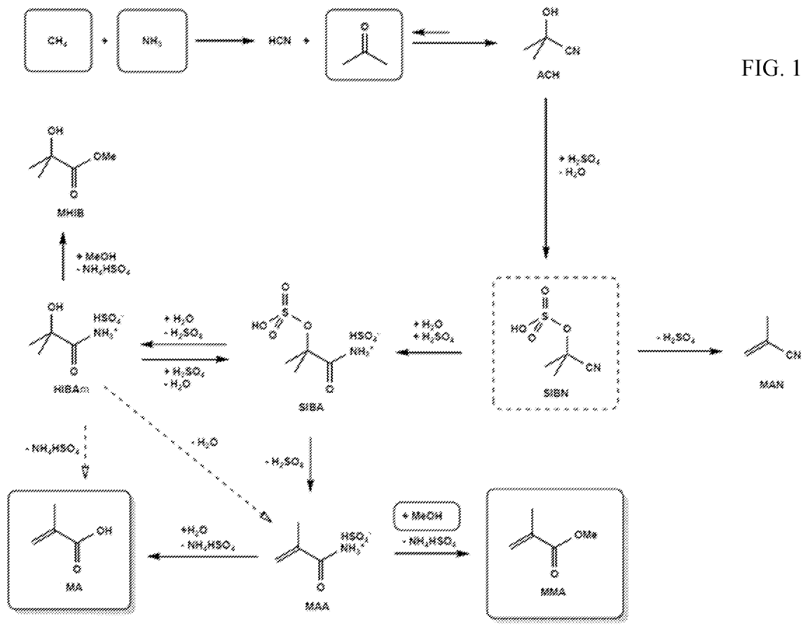

The abbreviations in FIG. 1 have the following meanings:
ACH acetone cyanohydrin;
SIBN alpha-sulfoxyisobutyronitrile;
SIBA alpha-sulfoxyisobutyramide;
SIBA·H₂SO₄ alpha-sulfoxyisobutyramide hydrogensulfate;
MAN methacrylonitrile;
HIBAm alpha-hydroxyisobutyramide;
HIBAm·H₂SO₄ alpha-hydroxyisobutyramide hydrogensulfate;
MAA methacrylamide;
MAA·H₂SO₄ methacrylamide hydrogensulfate;
MA methacrylic acid;
MMA methyl methacrylate;
HIBAc alpha-hydroxyisobutyric acid

FIG. 2 shows a flow diagram of a preferred embodiment of the second and third reaction stages (amidation and conversion, D to G) of the process according to the invention, and of the first reaction stage (A. B) and the workup (C) of the ACH prepared. Two successive process steps of amidation and conversion comprising the first amidation (D) and the first conversion (E), and the second amidation (F) and the second conversion (G), are shown.

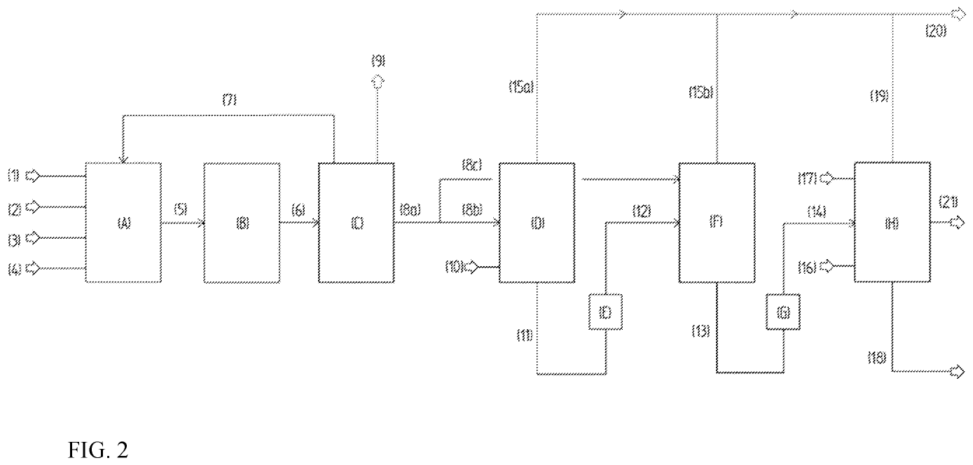

In FIG. 2, the reference symbols have the following meanings:

Apparatuses
(A) ACH synthesis reactor 1
(B) ACH synthesis reactor 2
(C) ACH distillation, workup
(D) amidation reactor (first reactor 1)
(E) first converter
(F) amidation reactor (second reactor 1)
(G) second converter
(H) hydrolysis or esterification

Streams of Matter
(1) acetone feed (acetone reactant)
(2) hydrogen cyanide feed
(3) diethylamine feed
(4) sulfuric acid feed
(5) synthesis mixture
(6) first reaction mixture, crude ACH to distillation
(7) low boiler recycle stream
(8a) pure ACH mixture
(8b) ACH feed to 1st stage (first substream of pure ACH mixture)
(8c) ACH feed to 2nd stage (second substream of pure ACH mixture)
(8) degassed amide mixture
(9) offgas from ACH distillation
(10) sulfuric acid feed
(11) stirred-up mixture, 1st stage
(12) intermediately converted reaction mixture
(13) stirred-up mixture, 2nd stage
(14) converted reaction mixture (third reaction mixture)
(15a) offgas from stage 1 amidation reactors
(15b) offgas from stage 2 amidation reactors
(16) optional methanol feed
(17) water feed
(18) cleavage acid
(19) esterification offgas
(20) overall offgas
(21) pure MA or MMA

### EXAMPLES

The preparation of methacrylamide in sulfuric acid solution, comprising the preparation of acetone cyanohydrin in a first reaction stage, the reaction thereof with sulfuric acid in the amidation of the second reaction stage, the thermal reaction of the second reaction mixture in the conversion of the third reaction stage, and the subsequent esterification with methanol and water in the third reaction stage was effected by the embodiment according to FIG. 2.

There follows a comparison of five inventive examples (Examples 1 to 5) using varying water contents in the ACH (8a, 8b, 8c) that was obtained in the first reaction stage (A, B) and the workup (C) and was fed to the second reaction stage (D, F), and in the sulfuric acid (10) in the feed to the amidation (D), with four comparative examples (Examples 6 to 9).

To ascertain the respective amidation yield, samples were taken downstream of the second conversion reactor (G). The concentrations of MAA, MA and HIBAm ascertained after quantification by means of HPLC were used for the mass balance of the process steps of the amidation (second reaction stage, D, F) and the conversion (third reaction stage, E, G). Results based on the individual process steps are shown in Tables 1 to 8 for the comparative examples and the inventive examples. In the listing of the measurement results, a measurement error in the HPLC analysis of ±0.2% is disclosed in each case.

The third reaction mixture (14) comprising MAA, MA and HIBAm obtained from the conversion (G) was subsequently admixed and esterified with methanol and water in a cascade of multiple esterification reactors III (H); in a separation column on top. MMA-containing crude mixture was withdrawn as vapours, condensed (21) and separated into an organic phase and an aqueous phase. MMA was obtained by workup of the organic phase.

The water content in the sulfuric acid feeds (4, 10) was determined by mass balance based on the sulfuric acid content in the streams, and the sulfuric acid content was ascertained by measuring the density and the speed of sound. The water content of the acetone feed (1) was determined by gas chromatography using a thermal conductivity detector. The water content of the hydrogen cyanide feed (2) was ascertained by Karl Fischer titration, and the water content of the pure ACH mixture (8a) was determined by mass balance based on the ACH content, with the ACH content ascertained by means of HPLC.

### Inventive Example 1

Methacrylamide was prepared from acetone cyanohydrin and sulfuric acid having a total water content of 18.48 mol % in the feed (8a, 10) from the amidation in the second reaction stage (D, F), based on the total amount of ACH fed into the second reaction stage (8a). The individual water contents of the ACH (8a/8b/8c) and sulfuric acid (10) feedstocks are shown in Table 10.

9500 kg/h of an acetone reactant (1) having a water content according to Table 10 was reacted with 4439 kg/h (2) of hydrogen cyanide (HCN) that had a water content of about 0.03% by weight, based on the overall stream (2), and 8 kg/h of diethylamine (3) as catalyst in reactor (A) in the liquid phase. In addition, a circulation distillate stream (7) from the ACH distillation (C) that contained ACH, acetone and HCN was recycled into reactor (A).

Reactor (A) is executed as a loop reactor with downstream delay vessel (B), with removal of the heat of reaction released in reactor (A) by means of cooling water via a shell-and-tube heat exchanger. The reactor was operated at 35 to 40° C. and standard pressure.

The synthesis gas mixture (5) at about 40° C. which was obtained in reactor (A), containing more than 85% by weight of ACH, based on the overall stream (5), was fed continuously to the downstream delay vessel (B) in which the synthesis mixture (5) matured. The delay vessel (B) is executed in the form of a cooled reservoir vessel with a pumped circulation system and was operated at about 10° C. and standard pressure.

The crude ACH obtained downstream of the delay vessel (B) (first reaction mixture, 6) with an elevated ACH content of 92.7% by weight, based on the overall stream (6), was subsequently fed continuously to a distillation step (C). For stabilization of the ACH obtained and for neutralization of the diethylamine catalyst still present, 32 kg/h of 98% sulfuric acid (4) was fed into the crude ACH (first reaction mixture, 6) via a mixing zone. The result was a stabilized crude ACH stream (6a).

The stabilized crude ACH stream (6a) was fed in continuously at the top of a distillation column (C). The distillation column (C) was operated as a stripping column at about 120 mbar, heated indirectly with hot steam (10 bar) and separated acetone, HCN and further low-boiling by-products from a pure ACH mixture (8a) as bottom product that contained 98.5% by weight of ACH, based on the overall stream (8a), and water and acetone as by-products. The correspondingly obtained composition can be found in Table 10.

The low-boiling distillate from distillation (C) was returned continuously to the first reaction stage (A) together with a vacuum pump condensate obtained as low-boiling recycle stream (7).

The output air (9) that was obtained in the vacuum station of the distillation (C) was removed continuously from the process and sent to controlled incineration.

The pure ACH mixture (8a) obtained, at a mass flow rate of 13 850 kg/h, was cooled down to 10° C. downstream of the distillation (C). Subsequently, the overall stream of the pure ACH mixture (8a) was divided into two substreams (8b, 8c) which, in the second reaction stage, were fed into the first reactor I (D) or into the second reactor I (F) for the amidation.

Substream 8b, at a mass flow rate of 9000 kg/h, was applied to the first amidation reactor I (D) together with the sulfuric acid stream (10) that had a total mass flow rate of 22 440 kg/h, and the composition of which is shown in Table 10. Sulfuric acid stream 10 was admixed with 50 ppm of phenothiazine as stabilizer before entering the first amidation reactor I.

The first amidation reactor I (D) is designed as a loop reactor and was operated at 98° C. Substream (8b) was fed to the first amidation reactor I (D) continuously and at a temperature of 20° C.

The amount of sulfuric acid (in stream 10) needed for the optimal conversion of the ACH in the first reactor I (D) and the second reactor I (F) was fed into the first reactor I (D) in a mass ratio to the total amount of ACH in the feed (8b+8c) of 1.62 kg_{H2SO4}/kg_{ACH} or 1.41 mol_{H2SO4}/mol_{ACH}.

A hot stirred-up mixture (11) at 98° C. was obtained from the first reactor I (D), which contained sulfoxyisobutyramide (SIBA), methacrylamide (MAA) and hydroxyisobutyramide (HIBAm), each dissolved in sulfuric acid. The stirred-up mixture (11), after gas separation, was fed continuously to an intermediate conversion in the first converter (E). The pressure differential required for conveying was implemented by means of the reactor circulation pump of the amidation reactor (D). The resultant offgas (15a) was removed from the process in the direction of the amidation output air (20).

The first converter (E) is executed as a flow tube reactor comprising a preheater segment and a delay segment. The stirred-up mixture (11) entering the first converter (E) was heated to 130° C. in the preheater segment. This was followed by further conversion in the delay segment.

The reaction mixture (12) exiting from the first converter (E) was then fed into the second amidation reactor I (F). The second amidation reactor (F) is constructed as a loop reactor analogously to the first amidation reactor I (D) and was likewise operated at about 98° C. The ACH-containing substream 8c at 4850 kg/h which is required in the second amidation reactor I (F) was introduced directly into the reaction mixture (12) in the second amidation reactor I (F) as well. Resultant offgas (15b) was removed from the process in the direction of the overall offgas (20).

For final conversion of the active constituents of the reaction mixture (13) that was obtained from the second amidation reactor I (F), a second conversion step was subsequently conducted in the second converter (G).

The second converter (G) is likewise executed as a flow tube reactor and comprises a preheater segment and a delay segment.

The reaction mixture (13) that entered the second converter (G) was first heated in the preheater segment to an optimal temperature shown in Table 10. The temperature to be established in the second converter (G) was ascertained by preliminary experiments and led to a maximum conversion of SIBA and HIBAm to MAA.

Subsequently, the reaction mixture (13) was converted further in the delay segment of the second converter (D), while maintaining the temperature established beforehand.

The reaction mixture obtained from the second converter (D) was then separated from gaseous secondary components that were removed in the form of an offgas stream (15c). The resulting third reaction mixture (14) was withdrawn continuously in liquid form from the second converter (G). The third reaction mixture (14) contained the components methacrylamide (MAA), methacrylic acid (MA) and hydroxyisobutyramide (HIBAm) according to Table 10. The respective HPLC analysis for determination of the respective components was conducted in triplicate; the respective arithmetic averages are entered in Table 1. Sampling and analysis were effected twice per day, with sampling on five successive days in total in steady-state operation of the plant.

The resultant overall yield of components (methacrylamide, methacrylic acid) which are convertible to the target product (methyl methacrylate) and were subsequently sent to an esterification (H) for preparation of methyl methacrylate is likewise shown in Table 1. The concentrations reported are based on the overall flow rate of the third reaction mixture (14). The average yield of 10 representative samples measured in steady-state operation of a process with the conditions specified was 93.0%. In stream (14), by means of HPLC, a MAN content of 120 ppm was measured.

TABLE 1
Results of Inventive Example 1

| | | Concentration in the product stream [% by wt.] | | | Yield [%] |
|---|---|---|---|---|---|
| | | | Methacrylic | | Methacrylamide + |
| Sample | Time | Methacrylamide | acid | Hydroxyisobutyramide | methacrylic acid |
| 1 | Day 1; 10:10 | 33.81 | 1.20 | 1.60 | 93.04 |
| 2 | Day 1; 13:40 | 33.90 | 1.15 | 1.63 | 93.29 |
| 3 | Day 2; 07:30 | 33.72 | 1.15 | 1.55 | 92.79 |
| 4 | Day 2; 10:26 | 33.78 | 1.12 | 1.58 | 92.96 |
| 5 | Day 3; 09:05 | 33.80 | 1.17 | 1.56 | 93.01 |
| 6 | Day 3; 11:08 | 33.98 | 1.17 | 1.54 | 93.51 |
| 7 | Day 4; 10:34 | 34.00 | 1.12 | 1.53 | 93.56 |
| 8 | Day 4; 14:11 | 33.71 | 1.12 | 1.51 | 92.76 |
| 9 | Day 5; 07:46 | 33.76 | 1.18 | 1.62 | 92.90 |
| 10 | Day 5; 09:19 | 33.40 | 1.19 | 1.81 | 91.91 |

| | |
|---|---|
| Average | 93.0 Range of |

variation of the
yield 1.7%

### Inventive Example 2

Methacrylamide was prepared from acetone cyanohydrin and sulfuric acid having a total water content of 3.66 mol % in the feed (8a, 10) from the amidation in the second reaction stage (D, F), based on the total amount of ACH fed into the second reaction stage (8a). The individual water contents of the ACH (8a/8b/8c) and sulfuric acid (10) feedstocks are shown in Table 10.

The process conditions with varied water content with regard to the ACH (8a) and sulfuric acid (10) feedstocks in the second reaction stage (D, F) are shown in Tables 2 and 10 respectively. Unless stated otherwise, the process was conducted under identical process conditions to those in Inventive Example 1.

The variation in the water content in the feed (8a,10) to the amidation (D, F) resulted in reaction conditions, the effect of which on the concentration in the third reaction mixture (14) downstream of the second converter (G) is summarized in Table 2. The resulting yield is likewise shown in Tables 2 and 10.

The average yield of 10 representative samples measured in steady-state operation of the process by means of HPLC, under the conditions specified, was 94.5%.

TABLE 2
Results of Inventive Example 2

| | | Concentration in the product stream [% by wt.] | | | Yield [%] |
|---|---|---|---|---|---|
| | | | Methacrylic | | Methacrylamide + |
| Sample | Time | Methacrylamide | acid | Hydroxyisobutyramide | methacrylic acid |
| 1 | Day 1; 16:10 | 34.66 | 1.03 | 1.03 | 94.49 |
| 2 | Day 1; 19:50 | 34.51 | 1.05 | 1.08 | 94.08 |
| 3 | Day 2; 07:20 | 34.72 | 1.05 | 0.98 | 94.65 |
| 4 | Day 2; 11:26 | 34.78 | 1.02 | 1.03 | 94.82 |
| 5 | Day 3; 07:05 | 34.80 | 1.04 | 1.04 | 94.87 |
| 6 | Day 3; 10:09 | 34.68 | 1.07 | 1.04 | 94.54 |
| 7 | Day 4; 08:30 | 34.50 | 1.01 | 1.01 | 94.05 |
| 8 | Day 4; 14:11 | 34.71 | 1.02 | 1.06 | 94.63 |
| 9 | Day 5; 07:30 | 34.76 | 1.03 | 0.96 | 94.76 |
| 10 | Day 5; 10:29 | 34.40 | 0.90 | 1.00 | 93.78 |

| | |
|---|---|
| Average | 94.5% Range of variation |

of the yield 1.1%

### Inventive Example 3

Methacrylamide was prepared from acetone cyanohydrin and sulfuric acid having a total water content of 0.47 mol % in the feed (8a, 10) from the amidation in the second reaction stage (D, F), based on the total amount of ACH fed into the second reaction stage (8a). The individual water contents of the ACH (8a/8b/8c) and sulfuric acid (10) feedstocks are shown in Table 10.

The process conditions with varied water content with regard to the ACH (8a) and sulfuric acid (10) feedstocks in the second reaction stage (D, F) are shown in Tables 3 and 10 respectively. Unless stated otherwise, the process was conducted under identical process conditions to those in Inventive Example 1.

The variation in the water content in the feed (8a,10) to the amidation (D, F) resulted in reaction conditions, the effect of which on the concentration in the third reaction mixture (14) downstream of the second converter (G) is summarized in Table 3. The resulting yield is likewise shown in Tables 3 and 10.

The average yield of 10 representative samples measured in steady-state operation of the process by means of HPLC, under the conditions specified, was 92.2%.

TABLE 3
Results of Inventive Example 3

| | | Concentration in the product stream [% by wt.] | | | Yield [%] |
|---|---|---|---|---|---|
| | | | Methacrylic | | Methacrylamide + |
| Sample | Time | Methacrylamide | acid | Hydroxyisobutyramide | methacrylic acid |
| 1 | Day 1; 09:05 | 35.01 | 1.21 | 0.06 | 92.18 |
| 2 | Day 1; 10:50 | 34.80 | 0.93 | 0.08 | 91.63 |
| 3 | Day 2; 07:30 | 35.31 | 0.86 | 0.05 | 92.97 |
| 4 | Day 2; 10:15 | 34.70 | 0.89 | 0.03 | 91.36 |
| 5 | Day 3; 07:20 | 35.26 | 0.98 | 0.08 | 92.84 |
| 6 | Day 3; 10:19 | 34.93 | 0.96 | 0.03 | 91.97 |
| 7 | Day 4; 08:22 | 35.05 | 0.99 | 0.03 | 92.29 |
| 8 | Day 4; 11:04 | 35.08 | 0.95 | 0.06 | 92.36 |
| 9 | Day 5; 07:35 | 34.78 | 1.01 | 0.08 | 91.57 |
| 10 | Day 5; 10:29 | 35.29 | 0.97 | 0.07 | 92.92 |

| | |
|---|---|
| Average | 92.2% Range of variation |

of the yield 1.6%

### Inventive Example 4

Methacrylamide was prepared from acetone cyanohydrin and sulfuric acid having a total water content of 15.67 mol % in the feed (8a, 10) from the amidation in the second reaction stage (D, F), based on the total amount of ACH fed into the second reaction stage (8a). The individual water contents of the ACH (8a/8b/8c) and sulfuric acid (10) feedstocks are shown in Table 10.

The process conditions with varied water content with regard to the ACH (8a) and sulfuric acid (10) feedstocks in the second reaction stage (D, F) are shown in Tables 4 and 10 respectively. Unless stated otherwise, the process was conducted under identical process conditions to those in Inventive Example 1.

The variation in the water content in the feed (8a,10) to the amidation (D, F) resulted in reaction conditions, the effect of which on the concentration in the third reaction mixture (14) downstream of the second converter (G) is summarized in Table 4. The resulting yield is likewise shown in Tables 4 and 10.

The average yield of 10 representative samples measured in steady-state operation of the process by means of HPLC, under the conditions specified, was 94.1%.

TABLE 4
Results of Inventive Example 4

| | | Concentration in the product stream [% by wt.] | | | Yield [%] |
|---|---|---|---|---|---|
| | | | Methacrylic | | Methacrylamide + |
| Sample | Time | Methacrylamide | acid | Hydroxyisobutyramide | methacrylic acid |
| 1 | Day 1; 10:05 | 34.49 | 1.13 | 1.12 | 94.49 |
| 2 | Day 1; 12:50 | 34.30 | 1.15 | 1.11 | 93.97 |
| 3 | Day 2; 08:30 | 34.32 | 1.05 | 1.06 | 94.02 |
| 4 | Day 2; 11:30 | 34.33 | 1.13 | 1.08 | 94.05 |
| 5 | Day 3; 07:10 | 34.31 | 1.10 | 1.12 | 94.00 |
| 6 | Day 3; 10:20 | 34.38 | 1.07 | 1.11 | 94.19 |
| 7 | Day 4; 08:20 | 34.28 | 1.11 | 1.06 | 93.91 |
| 8 | Day 4; 11:22 | 34.28 | 1.12 | 1.11 | 93.91 |
| 9 | Day 5; 07:30 | 34.29 | 1.17 | 1.12 | 93.94 |
| 10 | Day 5; 10:20 | 34.30 | 1.06 | 1.09 | 93.97 |

| | |
|---|---|
| Average | 94.1% Range of variation |

of the yield 0.6%

### Inventive Example 5

Methacrylamide was prepared from acetone cyanohydrnn and sulfuric acid having a total water content of 5.62 mol % in the feed (8a, 10) from the amidation in the second reaction stage (D, F), based on the total amount of ACH fed into the second reaction stage (8a). The individual water contents of the ACH (8a/8b/8c) and sulfuric acid (10) feedstocks are shown in Table 10.

The process conditions with varied water content with regard to the ACH (8a) and sulfuric acid (10) feedstocks in the second reaction stage (D. F) are shown in Tables 5 and 10 respectively. Unless stated otherwise, the process was conducted under identical process conditions to those in Inventive Example 1.

The variation in the water content in the feed (8a,10) to the amidation (D, F) resulted in reaction conditions, the effect of which on the concentration in the third reaction mixture (14) downstream of the second converter (G) is summarized in Table 5. The resulting yield is likewise shown in Tables 5 and 10.

The average yield of 10 representative samples measured in steady-state operation of the process by means of HPLC, under the conditions specified, was 96.7%.

TABLE 5
Results of Inventive Example 5

| | | Concentration in the product stream [% by wt.] | | | Yield [%] |
|---|---|---|---|---|---|
| | | | Methacrylic | | Methacrylamide + |
| Sample | Time | Methacrylamide | acid | Hydroxyisobutyramide | methacrylic acid |
| 1 | Day 1; 10:00 | 36.21 | 0.93 | 0.22 | 96.24 |
| 2 | Day 1; 13:50 | 36.30 | 0.94 | 0.26 | 96.69 |
| 3 | Day 2; 07:40 | 36.32 | 0.96 | 0.26 | 96.81 |
| 4 | Day 2; 11:23 | 36.34 | 0.92 | 0.30 | 96.84 |
| 5 | Day 3; 08:05 | 36.36 | 0.92 | 0.31 | 96.91 |
| 6 | Day 3; 11:05 | 36.40 | 0.92 | 0.29 | 96.90 |
| 7 | Day 4; 09:34 | 36.27 | 0.94 | 0.28 | 96.65 |
| 8 | Day 4; 13:10 | 36.35 | 0.95 | 0.26 | 96.71 |
| 9 | Day 5; 08:40 | 36.30 | 0.95 | 0.25 | 96.63 |
| 10 | Day 5; 11:18 | 36.37 | 0.93 | 0.22 | 96.81 |

| | |
|---|---|
| Average | 96.7 Range of variation |

of the yield 0.7%

### Comparative Example 6

Methacrylamide was prepared from acetone cyanohyddn and sulfuric acid having a total water content of 5.62 mol % in the feed (8a, 10) from the amidation in the second reaction stage (D, F), based on the total amount of ACH fed into the second reaction stage (8a). The individual water contents of the ACH (8a/8b/8c) and sulfuric acid (10) feedstocks are shown in Table 10.

The process conditions with varied water content with regard to the ACH (8a) and sulfuric acid (10) feedstocks in the second reaction stage (D. F) are shown in Table 10. Unless stated otherwise, the process was conducted under identical process conditions to those in Inventive Example 1.

In a departure from the above examples, the amidation reactors I (D) and (F) were operated at 80° C. rather than at 98° C. This much lower amidation temperature in the second reaction stage led to a significant increase in viscosity of the reaction mixture (11, 13), which immediately significantly reduced the circulation of the reaction medium in the first reactor I (D) and in the second reactor I (F) that was needed for heat exchange. This mode of operation then led to partial subcooling of the reaction medium in subregions of the cooling water-operated heat exchangers of the first reactor I (D) or of the second reactor I (F), and ultimately led to precipitation reactions. This blocked the plant, which had to be shut down.

For that reason, it was not possible to record any analysis data with regard to the concentrations in steady-state operation.

### Comparative Example 7

Methacrylamide was prepared from acetone cyanohydrin and sulfuric acid having a total water content of 20.66 mol % in the feed (8a, 10) from the amidation in the second reaction stage (D, F), based on the total amount of ACH fed into the second reaction stage (8a). The individual water contents of the ACH (8a/8b/8c) and sulfuric acid (10) feedstocks are shown in Table 10.

The process conditions with varied water content with regard to the ACH (8a) and sulfuric acid (10) feedstocks in the second reaction stage (D, F) are shown in Tables 6 and 10 respectively. Unless stated otherwise, the process was conducted under identical process conditions to those in Inventive Example 1.

The variation in the water content in the feed (8a,10) to the amidation (D, F) resulted in reaction conditions, the effect of which on the concentration in the third reaction mixture (14) downstream of the second converter (G) is summarized in Table 6. The resulting yield is likewise shown in Tables 6 and 10.

The average yield of 10 representative samples measured in steady-state operation of the process by means of HPLC, under the conditions specified, was 89.1%.

Very watery conditions existed in the feed (8a, 10) to the amidation, and even adjustment of the conversion conditions (cf. Table 10) achieved only a very low yield. The high water content in the amidation reactors (D, F) made it impossible to convert the HIBAm inevitably formed in the amidation (D, F) to MAA without breakdown of MAA already present.

TABLE 6
Results of Comparative Example 7

| | | Concentration in the product stream [% by wt.] | | | Yield [%] |
|---|---|---|---|---|---|
| | | | Methacrylic | | Methacrylamide + |
| Sample | Time | Methacrylamide | acid | Hydroxyisobutyramide | Methacrylic acid |
| 1 | Day 1; 08:05 | 32.57 | 1.71 | 2.30 | 89.92 |
| 2 | Day 1; 10:05 | 32.23 | 1.62 | 2.37 | 88.98 |
| 3 | Day 2; 07:45 | 32.39 | 1.63 | 2.39 | 89.42 |
| 4 | Day 2; 11:45 | 32.28 | 0.89 | 2.21 | 89.12 |
| 5 | Day 3; 08:15 | 31.92 | 1.77 | 2.80 | 88.13 |
| 6 | Day 3; 11:05 | 32.19 | 1.58 | 2.12 | 88.87 |
| 7 | Day 4; 08:32 | 31.90 | 1.62 | 2.74 | 88.07 |
| 8 | Day 4; 11:19 | 32.61 | 1.59 | 2.40 | 90.03 |
| 9 | Day 5; 08:21 | 32.54 | 1.62 | 2.30 | 89.84 |
| 10 | Day 5; 11:19 | 32.10 | 1.80 | 2.22 | 88.62 |

| | |
|---|---|
| Average | 89.1 Range of variation |

of the yield 2%

### Comparative Example 8

Methacrylamide was prepared from acetone cyanohydrnn and sulfuric acid having a total water content of 3.66 mol % in the feed (8a, 10) from the amidation in the second reaction stage (D, F), based on the total amount of ACH fed into the second reaction stage (8a). The individual water contents of the ACH (8a/8b/8c) and sulfuric acid (10) feedstocks are shown in Table 10.

The process conditions with varied water content with regard to the ACH (8a) and sulfuric acid (10) feedstocks in the second reaction stage (D, F) are shown in Tables 7 and 10 respectively. Unless stated otherwise, the process was conducted under identical process conditions to those in Inventive Example 1.

The variation in the water content in the feed (8a,10) to the amidation (D, F) resulted in reaction conditions, the effect of which on the concentration in the third reaction mixture (14) downstream of the second converter (G) is summarized in Table 7. The resulting yield is likewise shown in Tables 7 and 10.

The average yield of 10 representative samples measured in steady-state operation of the process by means of HPLC, under the conditions specified, was 89.9%.

Oleum (100.5% H₂SO₄, 0.5% free SO₃) was fed into the amidation in the sulfuric acid feed (10), and even by adjusting the conversion conditions (cf. Table 10) it was possible to achieve only a low yield. Surprisingly, it was not possible to achieve high yields in combination with oleum in the sulfuric acid feed (10), i.e. with a feed of water into the amidation (D, F) solely via the ACH feed (8a). A high water content in the ACH feed (8a) that led to a good yield in Inventive Examples 1 to 4, for example, was found to be disadvantageous in combination with oleum in the sulfuric acid feed (10).

TABLE 7
Results of Comparative Example 8

| | | Concentration in the product stream [% by wt.] | | | Yield [%] |
|---|---|---|---|---|---|
| | | | Methacrylic | | Methacrylamide + |
| Sample | Time | Methacrylamide | acid | Hydroxyisobutyramide | Methacrylic acid |
| 1 | Day 1; 07:00 | 34.10 | 0.29 | 0.23 | 90.49 |
| 2 | Day 1; 10:08 | 33.91 | 0.14 | 0.15 | 89.99 |
| 3 | Day 2; 08:40 | 33.73 | 0.15 | 0.15 | 89.51 |
| 4 | Day 2; 11:25 | 33.52 | 0.22 | 0.20 | 88.95 |
| 5 | Day 3; 07:23 | 33.49 | 0.26 | 0.21 | 88.87 |
| 6 | Day 3; 11:02 | 33.22 | 0.25 | 0.22 | 88.15 |
| 7 | Day 4; 07:49 | 34.06 | 0.26 | 0.25 | 90.38 |
| 8 | Day 4; 11:10 | 34.08 | 0.28 | 0.29 | 90.44 |
| 9 | Day 5; 08:13 | 34.29 | 0.24 | 0.22 | 90.99 |
| 10 | Day 5; 11:17 | 34.26 | 0.24 | 0.23 | 90.91 |

| | |
|---|---|
| Average | 89.9 Range of variation |

of the yield 2.4%

### Comparative Example 9

Methacrylamide was prepared from acetone cyanohydrin and sulfuric acid having a total water content of 0.47 mol % in the feed (8a, 10) from the amidation in the second reaction stage (D, F), based on the total amount of ACH fed into the second reaction stage (8a). The individual water contents of the ACH (8a/8b/8c) and sulfuric acid (10) feedstocks are shown in Table 10.

The process conditions with varied water content with regard to the ACH (8a) and sulfuric acid (10) feedstocks in the second reaction stage (D, F) are shown in Tables 8 and 10 respectively. Unless stated otherwise, the process was conducted under identical process conditions to those in Inventive Example 1.

The variation in the water content in the feed (8a,10) to the amidation (0, F) resulted in reaction conditions, the effect of which on the concentration in the third reaction mixture (14) downstream of the second converter (G) is summarized in Table 8. The resulting yield is likewise shown in Tables 8 and 10.

The average yield of 10 representative samples measured in steady-state operation of the process by means of HPLC, under the conditions specified, was 90.9%.

Low-water conditions existed in the ACH feed (8a, 10) to the amidation, and even adjustment of the conversion conditions (cf. Table 10) achieved only a low yield. It was not possible to compensate for the lack of water in the sulfuric acid feed (10) via the water content in the ACH feed (8a).

TABLE 8
Results of Comparative Example 9

| | | Concentration in the product stream [% by wt.] | | | Yield [%] |
|---|---|---|---|---|---|
| | | | Methacrylic | | Methacrylamide + |
| Sample | Time | Methacrylamide | acid | Hydroxyisobutyramide | methacrylic acid |
| 1 | Day 1; 07:22 | 34.85 | 0.04 | 0.02 | 91.60 |
| 2 | Day 1; 10:56 | 34.60 | 0.05 | 0.02 | 90.94 |
| 3 | Day 2; 08:07 | 34.73 | 0.04 | 0.02 | 91.28 |
| 4 | Day 2; 10:05 | 34.64 | 0.03 | 0.06 | 91.05 |
| 5 | Day 3; 07:03 | 34.32 | 0.00 | 0.04 | 90.21 |
| 6 | Day 3; 11:16 | 34.78 | 0.00 | 0.01 | 91.42 |
| 7 | Day 4; 09:49 | 34.77 | 0.03 | 0.00 | 91.39 |
| 8 | Day 4; 12:10 | 34.68 | 0.05 | 0.02 | 91.15 |
| 9 | Day 5; 08:41 | 34.29 | 0.04 | 0.03 | 90.13 |
| 10 | Day 5; 11:53 | 34.14 | 0.04 | 0.03 | 89.73 |

| | |
|---|---|
| Average | 90.9 Range of |

variation of the
yield 1.9%

### Comparative Example 10

Methacrylamide was prepared from acetone cyanohydrin and sulfuric acid having a total water content of 0.05 mol % in the feed (8a, 10) from the amidation in the second reaction stage (D, F), based on the total amount of ACH fed into the second reaction stage (8a). The individual water contents of the ACH (8a/8b/8c) and sulfuric acid (10) feedstocks are shown in Table 10.

The process conditions with varied water content with regard to the ACH (8a) and sulfuric acid (10) feedstocks in the second reaction stage (D, F) are shown in Tables 9 and 10. Unless stated otherwise, the process was conducted under identical process conditions to those in Inventive Example 1.

The variation in the water content in the feed (8a,10) to the amidation (D, F) resulted in reaction conditions, the effect of which on the concentration in the third reaction mixture (14) downstream of the second converter (G) is summarized in Table 9. The resulting yield is likewise shown in Tables 9 and 10.

The average yield of three representative samples measured in steady-state operation of the process by means of HPLC, under the conditions specified, was 89.5%.

Low-water conditions existed in the ACH feed (8a, 10) to the amidation, and even adjustment of the conversion conditions (cf. Table 10) achieved only a low yield. It was not possible to compensate for the lack of water in the sulfuric acid feed (10) via the water content in the ACH feed (8a). The further decrease in water content in the ACH feed (8a) compared to Comparative Example 9 resulted in another significant lowering of the yield.

TABLE 9
Results of Comparative Example 10

| | | Concentration in the product stream [% by wt.] | | | Yield [%] |
|---|---|---|---|---|---|
| | | | Methacrylic | | Methacrylamide + |
| Sample | Time | Methacrylamide | acid | Hydroxyisobutyramide | methacrylic acid |
| 2 | Day 1; 10:32 | 34.60 | 0.03 | 0.02 | 90.94 |
| 3 | Day 2; 08:11 | 34.53 | 0.04 | 0.02 | 91.28 |
| 4 | Day 2; 10:24 | 34.64 | 0.03 | 0.04 | 91.05 |

| | |
|---|---|
| Average | 89.5 |

Summary of the examples:

TABLE 10
Selected process parameters

| | Example 1 | Example 2 | Example 3 | Example 4 | Example 5 | Unit |
|---|---|---|---|---|---|---|
| Water content of acetone, based on stream 1 | 0.9 | 0.9 | 0.1 | 0.1 | 0.5 | % by wt. |
| H₂SO₄ concentration, based on stream 10 | 98.0 | 100.0 | 100.0 | 98.0 | 99.5 | % by wt. |
| Water content of sulfuric acid, based on stream 10 | 2.0 (10.0) | 0.0 (0) | 0.0 (0) | 2.0 (10.0) | 0.5 (2.7) | % by wt./(mol %) |
| ACH concentration, based on stream 8a | 98.5 | 98.5 | 99.2 | 99.2 | 98.9 | % by wt. |
| Water content in the ACH, based on stream 8a | 0.8 (3.66) | 0.8 (3.66) | 0.1 (0.47) | 0.1 (0.47) | 0.4 (1.7) | % by wt./(mol %) |
| H₂O from ACH (8a) and H₂SO₄ | 18.48 | 3.66 | 0.47 | 15.67 | 5.62 | mol % |
| (10) based on total amount of ACH (8a) | | | | | | |
| Amidation temperature in first reactor I (D) | 98 | 98 | 98 | 98 | 98 | ° C. |
| Amidation temperature in second reactor I (F) | 98 | 98 | 98 | 98 | 98 | ° C. |
| Optim. converter temperature (G) | 165 | 160 | 155 | 164 | 159 | ° C. |
| Molar H₂SO₄(10)/ACH(8a) ratio | 1.41 | 1.41 | 1.41 | 1.41 | 1.41 | mol/mol |
| ACH division (8b/8c) | 65/35 | 65/35 | 65/35 | 65/35 | 65/35 | kg/kg |
| Total mass flow rate of third reaction mixture (14) | 36243 | 36196 | 36058 | 36253 | 36266 | kg/h |
| Ø MAA concentration, based on stream 14 | 33.8 | 34.6 | 35.0 | 34.6 | 35.4 | % by wt. |
| Ø MA concentration, based on stream 14 | 1.2 | 1.0 | 0.2 | 1.1 | 1.1 | % by wt. |
| Ø HIBAm concentration, based on stream 14 | 1.6 | 0.5 | 0.1 | 1.1 | 0.2 | % by wt. |
| Average yield (methacrylamide + methacrylic | Ø 93.0 | Ø 94.5 | Ø 92.2 | Ø 94.1 | Ø 96.7 | % |
| acid in 14) based on ACH in 8a | | | | | | |
| Range of variation of the yield | 1.7 | 1.1 | 1.6 | 0.6 | 0.7 | % |
| | Comp. | Comp. | Comp. | Comp. | Comp. | |
| | Example 6 | Example 7 | Example 8 | Example 9 | Example 10 | Unit |
| Water content of acetone, based on stream 1 | 0.5 | 0.5 | 0.9 | 0.1 | 0.01 | % by wt. |
| H₂SO₄ concentration, based on stream 10 | 99.5 | 97.5 | 100.5 | 100.5 | 100.3 | % by wt. |
| Water content of sulfuric acid, based on stream 10 | 0.5 (2.7) | 2.5 (12.3) | 0.0 (0) | 0.0 (0) | 0.0 (0) | % by wt./(mol %) |
| ACH concentration, based on stream 8a | 98.9 | 98.9 | 98.5 | 99.2 | 99.2 | % by wt. |
| Water content in the ACH, based on stream 8a | 0.4 (1.7) | 0.4 (1.7) | 0.8 (3.66) | 0.1 (0.47) | 0.01 (0.05) | % by wt./(mol %) |
| H₂O from ACH (8a) and H₂SO₄ (10) | 5.62 | 20.66 | 3.66 | 0.47 | 0.05 | mol % |
| based on total amount of ACH (8a) | | | | | | |
| Amidation temperature in first reactor I (D) | 80 | 98 | 98 | 98 | 98 | ° C. |
| Amidation temperature in second reactor I (F) | 80 | 98 | 98 | 98 | 98 | ° C. |
| Optim. converter temperature (G) | 159 | 166 | 154 | 152 | 152 | ° C. |
| Molar H₂SO₄(10)/ACH(8a) ratio | 1.41 | 1.41 | 1.41 | 1.41 | 1.41 | mol/mol |
| ACH division (8b/8c) | 65/35 | 65/35 | 65/35 | 65/35 | 65/35 | kg/kg |
| Total mass flow rate of third reaction mixture (14) | — | 36166 | 35976 | 36021 | 35948 | kg/h |
| Ø MAA concentration, based on stream 14 | — | 32.4 | 34.1 | 34.8 | 34.6 | % by wt. |
| Ø MA concentration, based on stream 14 | — | 1.7 | 0.2 | 0.04 | 0.03 | % by wt. |
| Ø HIBAm concentration, based on stream 14 | — | 2.3 | 0.2 | 0.02 | 0.02 | % by wt. |
| Average yield (methacrylamide + methacrylic acid | — | Ø 89.1 | Ø 89.9 | Ø 90.9 | Ø 89.5 | % |
| in 14) based on ACH in 8a | | | | | | |
| Range of variation of the yield | — | 2.0 | 2.4 | 1.9 | 2.6 | % |

A comparison of Inventive Examples 1 to 5 emphasizes that a water content according to the invention in the amidation (D, F, 8a, 10), determined both by the water content of the acetone reactant (1) fed into the first reaction stage (A, B) and by the water content of the sulfuric acid (10) used in the amidation (0), is essential for high and stable yields. According to Table 10, a total amount of water between 0.5 and 18.5 mol %, based on the total amount of ACH fed into the second reaction stage, achieves the highest yields, provided that no oleum has been used in place of sulfuric acid (see Comparative Examples 8, 9 and 10), which worsened the yields further. Furthermore, the scatter of the yield measured was at its lowest, and hence the overall process was at its most stable, within a range from 6 to 16 mol % of the total amount of water, based on the total amount of ACH that was fed to the second reaction stage.

It was likewise found that, as well as the total amount of water that leads to achievement of the maximum yield, the concentration of sulfuric acid (10) fed in is of particular relevance (cf. Examples 2 and 5, and 2 and 8).

In addition, it was found that, in the case of exceedance of a total amount of water in the amidation of 16 mol %, based on a total amount of ACH fed into the second reaction stage, a distinct increase in the MA and HIBAm components is apparent in the third reaction mixture (14), which caused a loss of yield among other effects. Although it is possible to reduce the proportion of HIBAm by establishing a higher conversion temperature (E, G), with conversion to MAA, there are limits to this process. On comparison of Examples 4, 1 and 7, which represent a rising total water content from 5.6 to 18.5 and 20.6 mol %, higher conversion temperatures were used in order to convert the proportion of HIBAm that rose to an increasingly significant extent. This was no longer fully possible over and above a total water content of 16 mol % (cf. Examples 1 and 4), and ultimately led to loss of yield.

A comparison of Examples 2 and 8 follows on from this effect. Operation with 3.66 mol % of total water that was fed in exclusively with the ACH (8a) in both cases led to different yields here. In the case of 100% sulfuric acid (Example 2), the yield achieved is already at a good level, whereas the use of oleum (10) in Example 8 led to distinct yield losses. If the proportion of HIBAm and MA in the third reaction mixture (14) is considered in this connection, it is apparent that, in the case of 100% sulfuric acid (Example 2) at conversion temperature 160° C., a moderate proportion of HIBAm and MA has been produced with good yield. Even though these components occurred to a distinctly reduced degree in the case of the oleum method (100.5% in Example 8) and a conversion temperature of 152° C., a poorer overall yield was nevertheless found. If the product mass flow rates (14) of Examples 2 and 8 are compared, it becomes clear that the loss of yield arose from offgas losses (reduced product flow rate) and hence the formation of HIBAm was not solely responsible for the loss of yield. The use of oleum (10) in the case of a high water content in the ACH feed (8a) led to greater yield losses than was the case for 100% sulfuric acid (10).

1. A process for preparing methacrylic acid and/or alkyl methacrylate, the process comprising:
a. reacting acetone and hydrogen cyanide in the presence of a basic catalyst in a first reaction stage for synthesis of acetone cyanohydrin (ACH), to obtain a first reaction mixture comprising the acetone cyanohydrin (ACH);
b. working up the first reaction mixture comprising the acetone cyanohydrin (ACH);
c. reacting the acetone cyanohydrin (ACH) and sulfuric acid in one or more reactors I in a second reaction stage for amidation, at an amidation temperature in the range from 85° C. to 130° C., to obtain a second reaction mixture comprising sulfoxyisobutyramide and methacrylamide;
d. converting the second reaction mixture by heating to a conversion temperature in the range from 130° C. to 200° C., in one or more reactors II in a third reaction stage for conversion to obtain a third reaction mixture comprising predominantly methacrylamide (MAA) and sulfuric acid;
e. reacting the third reaction mixture with water and optionally alcohol, in one or more reactors III in a fourth reaction stage for hydrolysis or esterification, to obtain a fourth reaction mixture comprising the methacrylic acid and/or the alkyl methacrylate; and
f. optionally, separating the alkyl methacrylate from the fourth reaction mixture obtained from the fourth reaction stage;
wherein the sulfuric acid used in the second reaction stage has a concentration in the range from 98.0% by weight to 100.0% by weight;
wherein a pure ACH mixture which is fed to the second reaction stage has a water content within a range from 0.1 mol % to 10 mol %, based on the ACH present in the pure ACH mixture, and
wherein a total amount of water used in the second reaction stage is within a range from 0.1 mol % to 20 mol %, based on the ACH present in the pure ACH mixture.

2. The process according to claim 1, wherein the workup of the first reaction mixture in b. comprises a distillation step, wherein the acetone cyanohydrin (ACH) is separated at least partly from impurities and/or by-products that are lower-boiling than & acetone cyanohydrin (ACH).

3. The process according to either claim 1, wherein an acetone reactant which is used in the first reaction stage contains 0.1% by weight to 1% by weight of water, based on the overall amount of the acetone reactant.

4. The process according to claim 1, wherein a hydrogen cyanide reactant which is used in the first reaction stage contains 0.01% by weight to 0.1% by weight of water, based on the overall amount of the hydrogen cyanide reactant.

5. The process according to claim 1, wherein the total amount of the ACH fed to the second reaction stage is fed in with the pure ACH mixture.

6. The process according to claim 1, wherein the sulfuric acid and the acetone cyanohydrin (ACH) are used in the second reaction stage in a molar ratio of the sulfuric acid to the ACH in the range from 1.3 to 1.8, based on the total amount of ACH fed to the second reaction stage.

7. The process according to claim 1, wherein the third reaction mixture comprising the methacrylamide (MAA) contains not more than 3% by weight of g methacrylic acid (MA), not more than 2% by weight of alpha-hydroxyisobutyramide (HIBAm), and not more than 0.3% by weight of methacrylonitrile (MAN), based in each case on the overall amount of the third reaction mixture.

8. The process according to claim 1, wherein the third reaction mixture contains 30% by weight to 40% by weight of the methacrylamide (MAA), based on the overall amount of the third reaction mixture.

9. The process according to claim 1, wherein the second reaction stage comprises conversion of the acetone cyanohydrin (ACH) and the sulfuric acid in at least two separate reaction zones.

10. The process according to claim 1, wherein the second reaction stage comprises conversion of the acetone cyanohydrin (ACH) and the sulfuric acid in the one or more reactors I,
wherein the one or more reactors I comprises at least two separate reactors I,
wherein the sulfuric acid and the acetone cyanohydrin (ACH) are used in a first reactor I in a molar ratio of the sulfuric acid the ACH in the range from 1.6 to 3.0, based on an amount of the ACH used in the first reactor I, and
wherein the sulfuric acid and the acetone cyanohydrin (ACH) are used in a second reactor I in a molar ratio of the sulfuric acid to the ACH in the range from 1.2 to 2.0, based on the total amount of ACH fed into the second reaction stage.

11. The process according to claim 1, wherein the fourth reaction mixture comprising the alkyl methacrylate, which is obtained in the fourth reaction stage, is worked up in further steps comprising at least one distillation step and/or at least one extraction step.

12. The process according to claim 1, wherein the fourth reaction mixture comprising the alkyl methacrylate, which is obtained in the fourth reaction stage, is guided in gaseous form into a distillation step, wherein a tops fraction comprising the alkyl methacrylate, water, and alcohol, and a bottoms fraction comprising higher-boiling components are obtained, and
wherein the bottoms fraction is recycled fully or partly into the fourth reaction stage.

13. The process according to claim 12, wherein the tops fraction comprising the alkyl methacrylate, water, and alcohol is separated in a phase separation step into an organic phase comprising a predominant portion of the alkyl methacrylate and into an aqueous phase comprising the alcohol and further water-soluble compounds, and
wherein the aqueous phase is recycled fully or partly into the third reaction stage, and the organic phase comprising the predominant portion of the alkyl methacrylate is subjected to an extraction using water as extractant, wherein a further aqueous phase from this extraction is recycled into the third reaction stage.

14. The process according to claim 1, wherein the alkyl methacrylate is methyl methacrylate.

15. The process according to claim 1, wherein the alcohol in the third reaction mixture is methanol.

16. The process according to claim 1, wherein the pure ACH mixture has a water content within a range from 0.4 mol % to 5 mol %.

17. The process according to claim 1, wherein the total amount of water used in the second reaction stage is within a range from 0.4 mol % to 10 mol %.

18. The process according to claim 3, wherein the acetone reactant contains 0.1% by weight to 0.5% by weight of water.

19. The process according to claim 6, wherein in the second reaction stage, the molar ratio of the sulfuric acid to the ACH is in a range from 1.4 to 1.6.
